(19) United States
(12) Patent Application Publication
(10) Pub. No.: US 2009/0137390 A1
(43) Pub. Date: May 28, 2009

(54) Materials and methods for enhancing nitrogen fixation in plants

(76) Inventor: Eric Wendell Triplett, Gainesville, FL (US)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 11/630,844

(22) PCT Filed: Jun. 30, 2005
(86) PCT No.: PCT/US05/23535

§ 371 Date: Nov. 9, 2007

Related U.S. Application Data
(60) Provisional application No. 60/584,225, filed on Jun. 30, 2004.

(51) Int. Cl. A01N 63/00; A01H 5/00

(52) U.S. Cl. 504/117; 800/301

(57) ABSTRACT

The subject invention concerns materials and methods for providing or enhancing nitrogen fixation in plants. The invention provides for the use of nitrogen fixing bacteria that are isolated from nitrogen efficient plants. Plants for which enhanced nitrogen fixation is desired are inoculated with an effective amount of nitrogen firing bacteria of the invention. In an exemplified embodiment, the bacteria is Klebsiella Kp342. The subject invention also concerns means to increase the number of free-living nitrogen-fixing bacteria in plants. Mutants of beneficial endophytic bacteria that are resistant to plant defense responses can be used to colonize a plant in numbers higher than a wild type or a non-mutated bacteria can colonize a plant. The higher number of bacteria colonizing the plant provide for more nitrogen fixation for the plant. The subject invention concerns methods for producing non-leguminous plants that are capable e of utilizing atmospheric nitrogen by colonization with a nitrogen fixing endophyic bacteria that is resistant to plant defense responses. The subject invention also concerns the plants produced by the subject method. The subject invention also concerns methods for producing the mutant endophytic bacteria. The subject invention also concerns the mutant endophytic bacteria produced using the subject methods.

### CROSS-REFERENCE TO RELATED APPLICATION

This application claims the benefit of U.S. Provisional Application Ser. No. 60/584,225, filed Jun. 30, 2004.

### BACKGROUND OF THE INVENTION

Nitrogen gas (N₂) is a major component of the atmosphere of Earth. In addition, elemental nitrogen (N) is an important component of many chemical compounds which make up living organisms on Earth. Life forms, however, cannot use N₂ directly to synthesize the chemicals used in physiological processes, such as growth and reproduction. In order to utilize the N₂ in the chemicals of a life form, the N₂ must be combined with hydrogen. The combining of hydrogen with N₂ is referred to as nitrogen fixation. Nitrogen fixation, whether accomplished chemically or biologically, requires an investment of large amounts of energy. In biological systems, an enzyme known as nitrogenase catalyzes the reaction which results in nitrogen fixation.

An important goal of nitrogen fixation research is the extension of this phenotype to non-leguminous plants, particularly important agronomic grasses such as wheat, rice, and maize. Despite enormous progress in understanding the development of the nitrogen-fixing symbiosis between rhizobia and legumes, the path to use that knowledge to induce nitrogen-fixing nodules on non-leguminous crops is still not clear. Similarly, transforming plant genomes with bacterial nif genes to obtain nitrogen-fixing non-legumes remains a daunting task (Dixon et al., 1997).

Bacteria interact with plants in four ways, as pathogens, symbionts, epiphytes, or endophytes. Of these four types of bacterial-plant interactions, endophytic interactions are the least studied and least understood. Endophytes are defined here as bacteria that enter the interior of plants without causing disease symptoms or eliciting the formation of symbiotic structures. Endophytic bacteria are of agronomic interest because they can enhance plant growth and improve the nutrition of plants through nitrogen fixation (Boddey et al., 2003; Sevilla et al., 2001). They are also of medical interest because some bacterial endophytes are human pathogens that cannot be effectively removed by surface sterilization (Beuchat et al, 2001; Proctor et al., 2001; Taormina et al., 1999; Weissinger and Beuchat 2000; Weissinger et al., 2001). Nitrogen-fixing bacteria that inhabit the interior of grasses without causing any disease of symbiotic structures, called diazotrophic endophytes, are being investigated as to whether such bacteria can provide sufficient amounts of fixed nitrogen to relieve nitrogen deficiency in plants under conditions where N is limiting.

Definitive evidence that a particular bacterium is providing fixed N to the plant requires that: 1) total plant N must significantly increase upon inoculation preferably with a concomitant increase in N concentration in the plant; 2) nitrogen deficiency symptoms must be relieved under N-limiting conditions upon inoculation which should include an increase in dry matter; 3) N₂ fixation must be documented through the use of an ¹⁵N approach which can be isotope dilution experiments, ¹⁵N₂ reduction assays, or ¹⁵N natural abundance assays; 4) fixed N must be incorporated into a plant protein or metabolite; and 5) all of these effects must not be seen in uninoculated plants or in plants inoculated with a Nif mutant of the inoculum strain. In addition, the inoculum strain must be recovered from the host plant in order to fulfill Koch's postulates.

Previous attempts to demonstrate nitrogen fixation in wheat have shown little if any fixed N provided by diazotrophic bacteria. Rennie et al. (1983) used ¹⁵N isotope dilution to show that up to 32% of the N in wheat plants of one cultivar was derived from the atmosphere following inoculation with a strains of Bacillus polymyxa and Azospirillum brasilense but there was no increase in N concentration in the plants compared to the uninoculated control and there was no report of increased plant growth or a relief of nitrogen deficiency symptoms. Lethbridge and Davidson (1983) were unable to see N₂ fixation in some of the same wheat lines using some of the same bacteria as inoculants. Boddey et al. (1986a and 1986b) was also unable to observed fixed N in wheat from inoculation with Azospirillum strains. Kucey et al. (1988) observed small amounts of fixed N, up to 11% of plant N, in field grown wheat plants but the authors suggested that might be in error because the ¹⁵N was not uniformly distributed with depth as it was in this work. In all of these cases, Nif mutants were never used as controls. In Bremer et al. (1995), very little N₂ was fixed in wheat plants cultured in the greenhouse but these plants were not inoculated with any diazotrophs.

Recent studies have shown that inoculation with several bacterial endophytes on maize in greenhouse and field experiments failed to relieve nitrogen deficiency symptoms of the plants (Riggs et al., 2001). In previous work, different species or strains of enteric bacteria were found to differ greatly in their ability to colonize the interior of Medicago sativa (alfalfa) roots (Dong et al., 2003a). However, the mechanism of this strain specificity is not known. A strain isolated from maize, Klebsiella pneumoniae strain 342 (Kp342), colonizes the interior of several host plants in higher numbers than any other strain tested (up to 10⁷ cells per gram fresh weight (Dong et al., 2003a; Dong et al., 2003b). This strain, originally isolated from a nitrogen-efficient maize line (Chelius and Triplett 2000), fixes N₂ and increases maize yield in the field (Riggs et al., 2001). Kp342 also expresses nitrogenase in planta (Chelius and Triplett 2000) and occupies the interior of plants in much higher numbers than Klebsiella that were not of plant origin (Dong et al., 2003a). Fewer than ten cells of Kp342 are sufficient in the inoculum to fully colonize the plant (Dong et al., 2003a). Similarly various Salmonella strains differed in their ability to colonize alfalfa roots (Dong et al., 2003a).

In experiments with Gluconacetobacter diazotrophicus PA15 (Sevilla et al., 2001), ¹⁵N₂ was directly incorporated into the plants following inoculation with PA15 but not by inoculation with a nifD mutant. However, the authors did not determine whether fixed N was incorporated into a plant product. Also, the N concentration in the plant tissue did not increase significantly and the authors did not determine nitrogenase expression by the bacteria in planta.

Small amounts of nitrogen fixation may occur in Kallar grass upon inoculation with Azoarcus sp. BH72 (Hurek et al., 2002). Although dry matter and total N increases in BH72-inoculated plants were observed compared to the nifK mutant control, the nitrogen concentration in the plant actually decreased with BH72 inoculation. No evidence was presented to show that BH72 could relieve nitrogen deficiency symptoms. A decline in ¹⁵N natural abundance was observed in BH72-inoculated plants compared to the controls as expected if nitrogen fixation was occurring but this was only significant in roots, and not shoots. Natural abundance changes in ¹⁵N were not measured in any plant product and the authors were unable to confirm Koch's postulates as they failed to re-isolate BH72 after inoculation. So although fixed N may have been provided to Kallar grass by BH72, the amounts were just 1.4 mg N/plant for two-month old plants and not sufficient to significantly improve the nutrition of the plant. The increases in total N in this work were 30-45 mgN/plant with six-week old plants.

### BRIEF SUMMARY OF THE INVENTION

The subject invention concerns materials and methods for providing or enhancing nitrogen fixation in plants. The present invention provides for the use of nitrogen fixing bacteria that are isolated from nitrogen efficient plants. Plants that tend to be nitrogen inefficient or plants that are to be grown in nitrogen deficient soil can be inoculated with an effective amount of nitrogen fixing bacteria of the invention. In one embodiment, nitrogen fixation in a plant is provided upon inoculation with the nitrogen-fixing bacterium, Klebsiella pneumoniae 342 (Kp342). In an exemplified embodiment Kp342 bacteria relieved nitrogen deficiency symptoms and increased total N in a plant and increased N concentration in the plant. The subject invention also concerns nitrogen fixing bacteria isolated from a nitrogen efficient plant.

The subject invention also concerns methods for producing plants that are capable of utilizing atmospheric nitrogen, the method comprising inoculation and colonization of a plant, plant tissue, or a plant seed with a nitrogen fixing endophytic bacteria that is resistant to plant defense responses. The subject invention also concerns the plants produced by the subject method.

The subject invention also concerns methods for producing mutant endophytic bacteria of the invention that are resistant to plant defense responses and that can fix nitrogen. In one embodiment, the bacterium is a mutant of Kp342. The subject invention also concerns the mutant endophytic bacteria produced using the subject methods.

The subject invention also concerns materials and methods for inducing defense responses in plants in order to reduce the number of pathogenic bacteria that colonize the plant. In one embodiment, the defense response is an ethylene-mediated defense response. The subject invention also concerns engineered plants in which ethylene-mediated defense responses are expressed or can be induced in the plant. In one embodiment, a plant is engineered to overexpress an npr1 gene.

### BRIEF DESCRIPTION OF THE DRAWINGS

The patent or application file contains at least one drawing executed in color. Copies of this patent or patent application publication with color drawings will be provided by the Patent Office upon request and payment of the necessary fee.

FIGS. 1A and 1B are photographs that show six week old spring wheat Triticum aestivum L. cv. Trenton inoculated with Klebsiella pneumoniae strain 342 (Kp342) and nifH mutant of Kp342 (nifH) grown in labeled (¹⁵NH₄NO₃ 10 mg/kg soil-mix) sand-perlite. The plants in the three pots on the left were inoculated with the nfiH mutant of Kp342 while the plants in the three pots on the right were inoculated with Kp342. FIG. 1C shows chlorophyll readings of 6-weeks-old spring wheat Triticum aestivum L. cv. Trenton obtained from Minolta SPAD 502 where the ratio of transmittance from two wavelength (650 nm/940 nm) creates an arbitrary unit related to chlorophyll content. Plants were inoculated with Kp342, the nifH mutant or uninoculated (Uninoc.). Treated plants were grown in labeled (¹⁵NH₄NO₃ 10 mg/kg soil-mix) sand-perlite (open column) or sand-vermiculite (closed column). The columns represent the mean SPAD readings. The bars represent the standard error. Least Significant Difference (LSD) statistical analysis was calculated to determine difference between treatments. These differences are represented by letters inside the columns.

FIGS. 2A-2H show Triticum aestivum L. cv. Trenton plants inoculated with Klebsiella pneumoniae strain 342 (Kp342) and compared to uninoculated plants or inoculated with a nifH mutant. Dry roots (open columns) and shoots (closed columns) from plants grown in labeled (¹⁵NH₄NO₃ 10 mg/kg soil-mix) sand-perlite (FIGS. 2A, 2C, 2E, and 2G) and sand-vermiculite (FIGS. 2B, 2D, 2F, and 2H) were used to estimate dry weights (FIGS. 2A and 2B) total N per plant (FIGS. 2C and 2D) and total N concentration in shoots (FIGS. 2E and 2F) and roots (FIGS. 2G and 2H) in dried tissue, 6-weeks post inoculation. The columns represent the mean of dry weight for plants grown in sand-perlite and sand-vermiculite (FIGS. 2A and 2B, respectively), and total nitrogen per plant grown in sand-perlite and sand-vermiculite (FIGS. 2B and 2C, respectively). The columns also represent the mean total N concentration per gram of dried shoot (FIG. 2E) and root (FIG. 2G) for plants grown in sand-perlite (FIG. 2F) and sand-vermiculite (FIG. 2H). The bars represent the standard error. Least Significant Difference (LSD) statistical analysis was calculated to determine difference between treatments. Letters inside the columns represents the LSD calculations (normal for roots and Italics for shoots).

FIGS. 3A and 3B show the percent ¹⁵N content in 6-weeks-old Triticum aestivum L. cv. Trenton grown in labeled (¹⁵NH₄NO₃, 11.7 atom % excess, 10 mg/kg soil-mix) sand-perlite (solid open and closed columns A and B) and sand-vermiculite (dotted open and closed columns A and B). The percent ¹⁵N was analyzed from dried and ground shoots (FIG. 3A) and roots (FIG. 3B). Plants were inoculated with Klebsiella pneumoniae strain 342 (Kp342), nifH mutant of Kp342 (nifH), or uninoculated (control). The columns represent the mean % ¹⁵N in the tissue. The bars represent the standard error. Significant differences are indicated by letters within the columns. FIG. 3C shows the phaeophytin molecule contains 4 N atoms. Any or all of these may be labeled with ¹⁵N. This represents the ratio of the % of pheophytin molecules that contain zero to four ¹⁵N atoms in the two treatments (Kp342/nifH) versus the number of ¹⁵N atoms observed in the pheophytin molecule by mass spectrometry.

FIGS. 4A-4E are photographs that show the comparison of GFP-labeled (green) K pneumoniae 342 wild type (FIGS. 4A and 4C) and GFP-labeled (green) Kp342 nifH) mutant (FIGS. 4B and 4D) of spring wheat Triticum aestivum L. cv. Trenton root colonization. Cross sections of spring wheat roots were examined (A and B) as well as lateral root emergence Bars (50 um) (FIGS. 4C and 4D). FIG. 4E shows the immunolocalization of NifH produced by GFP-labeled Kp342 in root cross section. Cells are seen in yellow as the fluorophores of NifH (red) and GFP-labeled Kp342 are colocalized (yellow) Bars (50 um).

FIG. 4F shows the number of CFU recovered from the interior of roots Triticum aestivum L. cv. Trenton. Plants were inoculated with Klebsiella pneumoniae strain 342 (closed columns) and nifH mutant of Kp342 (open column) at 10² and 10⁴ CFU/plant inoculum level. The columns represent the means of each treatment. Each treatment consists of four replicates and each replicate consists of four plants. The bars represent the standard errors about the mean; gfw, gram (fresh weight).

FIGS. 5A and 5B are photographs that show scanning laser confocal microscopy at 20× magnification of longitudinal sections of Medicago truncatula wild type (FIG. 5A) and sickle mutant (FIG. 5B) hypocotyls showing colonization by GFP-labeled Kp342. Sections were visualized 9 days after inoculation. The inoculum level was 10⁴ CFU/plant. Bars 50 μm. FIG. 5C shows the numbers of bacterial CFU recovered from interior of M. truncatula Gaerten cv. A17 wild type and sickle mutant plant tissues 7 days after inoculation. Two-day-old seedlings were inoculated with Kp342 at different inoculum levels. Data points represent the means and the bars represent the standard errors about the mean resulting from four replicates with each replicate consisting of four plants.

FIG. 6 shows a number of CFU recovered from the interior of Medicago sativa (closed columns) or M. truncatula (open columns) roots and hypocotyls were determined 5 and 7 days post inoculation respectively. Seedlings of M. truncatula were inoculated with 10² CFU of Kp342 in the presence and absence of 1 ppm of the ethylene ation inhibitor, 1-MCP. Seedlings of M. sativa were inoculated with Kp342, 14028, the spaS mutant of 14028, the spaS mutant complemented with the spaS gene, the sipB mutant, and the sipB mutant complemented with the sipB gene, and the double flagellin mutant with insertions in fliC and fljB. Treatments included an untreated control, application of the ethylene precursor, 5 μM ACC, or treatment with the ethylene action inhibitor, 1 ppm 1-MCP. The bars represent the standard errors of the mean resulting from four replicates, each replicate consisting of four plants.

FIG. 7 shows the effect of ACC on endophytic colonization over time. The number of CFU recovered from the interior of Medicago truncatula roots and hypocotyls was determined each day for six days after inoculation with 10² cells of Kp342 per plant. Plants were treated with and without ACC (5 μM) at the time of inoculation. The columns represent the mean CFU recovered from the plants, and the bars represent the standard errors of the means resulting from four replicate treatments; gfw, gram (fresh weight). ACC treatments are statistically different from the controls on days 4, 5, and 6 at the 5% level of confidence.

FIG. 8 shows endophytic colonization of Medicago truncatula roots and hypocotyls treated with C₂H₄ on successive days. Medicago truncatula seedlings were inoculated with 10² cells per plant of Kp342. ACC (5 μM) was used as a control on day 0 to show that the effects of ACC and C₂H₄ are similar. C₂H₄ (5 μM) was applied to different sets of plants beginning one day prior to inoculation (Day 1) and continuing each day up to 6 days after inoculation. The columns represent the mean CFU recovered from the plants 7 days post inoculation. The bars represent the standard errors of the means resulting from four replicate treatments; gfw, gram (fresh weight). Asterisks represent differences that are statistically significant from plants treated with C₂H₄ at day 0 at the 5% level of confidence.

FIG. 9 shows endophytic colonization of Triticum asetivum roots in the presence of increasing concentrations of ACC. Number of CFU recovered from the interior of the roots and hypocotyls of wheat seedlings. Roots of one-day old seedlings were inoculated with 10⁴ cells of 14028 (diamonds) and 10² cells of Kp342 (squares). Plants were harvested five days after inoculation. The data points represent the means and the bars represent the standard errors of the means resulting from four replicate treatments.

FIG. 10 shows a number of CFU recovered from the interior of wheat roots and hypocotyls. Roots of one-day old seedlings were inoculated with 10⁴ cells of 14028, the sipB mutant of 14028. The sipB mutants complemented with sipB gene, and the double flagellin mutant (fliC/fliB) of 14028. Columns represent the means of each treatment and the bars represent the standard errors of the means resulting from four replicate treatments; gfw, gram (fresh weight).

FIG. 11 shows root endophytic colonization of three Arabidopsis thaliana genotypes inoculated with 14028, the flagella mutant of 14028, the sipB mutant of 14028, the complemented sipB mutant, and Kp342. Number of CFU recovered from the interior of roots of A. thaliana cv. wild type, nahG, and npr1. The columns represent the means of each treatment. Each treatment consists of four replicates and each replicate consists of four plants. The bars represent the standard errors about the mean; gfw, gram (fresh weight). The letters in each column represent statistical differences with respect to the wild-type plant. The asterisks represent statistical differences with respect to the wild-type plant inoculated with 14028.

FIGS. 12A-12H are photographs that show histochemical assays of Arabidopsis thaliana PRl::GUS (FIGS. 12A-12G) and of A. thaliana wild type (FIG. 12H). The treatments were: uninoculated (FIG. 12A); inoculation with H₂O (FIG. 12B); sprayed with 5 mM salicylic acid (FIG. 12C); leaves infiltrated with 10⁷ CFU of P. syringae DC3000 (FIG. 12D); root inoculation with 14028 (FIG. 12E); root inoculation with the sipB mutant of 14028 (FIG. 12F); root inoculation with the sipB mutant complemented with the sipB gene (FIG. 12G); and uinoculated wild type A. thaliana (FIG. 12H).

FIG. 13 shows a model for the regulation of the endophytic colonization of plants by enteric bacteria.

### BRIEF DESCRIPTION OF THE SEQUENCES

SEQ ID NO: 1 is a PCR primer that can be used according to the subject invention.

SEQ ID NO: 2 is a PCR primer that can be used according to the subject invention.

SEQ ID NO: 3 is a polynucleotide encoding an NPR1 polypeptide.

SEQ ID NO: 4 is the NPR1 polypeptide encoded by SEQ ID NO: 3.

SEQ ID NO: 5 is a polynucleotide encoding an NPR1 polypeptide.

SEQ ID NO: 6 is the NPR1 polypeptide encoded by SEQ ID NO: 5.

SEQ ID NO: 7 is a polynucleotide encoding an NPR1 polypeptide.

SEQ ID NO: 8 is the NPR1 polypeptide encoded by SEQ ID NO: 7.

SEQ ID NO: 9 is a polynucleotide encoding an NPR1 polypeptide.

SEQ ID NO: 10 is a polynucleotide encoding an NIF polypeptide.

### DETAILED DISCLOSURE OF THE INVENTION

The subject invention concerns materials and methods for providing for or enhancing nitrogen fixation in plants. The invention provides for the use of nitrogen fixing endophytic bacteria that are originally isolated from a nitrogen efficient plant. In one embodiment, plants for which enhanced nitrogen fixation is desired are inoculated with an effective amount of nitrogen fixing bacteria of the invention. The plant or plant tissue thereof can be inoculated with the nitrogen fixing bacteria. In one embodiment, plant parts, such as roots, are inoculated with bacteria for colonization of the plant. In an exemplified embodiment, plant seeds are inoculated with nitrogen fixing bacteria of the invention. In another embodiment, the bacteria of the invention are seed borne and thus are present in the seed obtained from a plant already colonized by the bacteria. Thus, seed from a plant colonized by the nitrogen fixing bacteria can be grown to produce a plant that is itself colonized with the bacteria, thereby avoiding the process of inoculating the seed or plant at the time of planting or after planting. In one embodiment, the plant is a non-leguminous plant, such as an agronomically important grass, e.g., wheat, rice, maize, barley, oats, sorghum, and rye.

In one embodiment, the nitrogen fixing bacteria of the present invention is Klebsiella pneumoniae. In an exemplified embodiment, the bacteria is Klebsiella Kp342. In one embodiment of the invention, the nitrogen fixing bacteria are resistant to a defense response of a plant. In a specific embodiment, the nitrogen fixing bacteria do not express, and/or express lower levels of, one or more extracellular components, such as flagella or secretion systems. In one embodiment, the bacteria do not express the gene or gene product from one or more of a nip, spa, or fli gene. In another embodiment, the bacteria express a mutant nonfunctional gene or gene product from one or more of a sip, spa, or fli gene. In a specific embodiment, the sip gene is sipB, the spa gene is spaS, and the fli gene is fliC or fliB gene.

In a further embodiment, the plants used in the present invention are resistant to colonization or infection by a bacterial pathogen. In one embodiment, plants are engineered to express defense responses. In another embodiment, plants are engineered wherein defense responses can be induced upon exposure of the plant to a substance or condition. The defense response can be, for example, an ethylene-mediated defense response. In one embodiment, the defense responses can be salicylic acid-mediated (SA-mediated) or salicylic acid-independent (SA-independent) responses. In a specific embodiment, a plant is engineered to overexpress an NPR1 gene. In a specific embodiment, the plant is resistant to Salmonella sp.

Nitrogen fixation in wheat by Kp342 that meets all of the criteria for such experiments as outlined in the Background section is demonstrated herein. Compared to the uninoculated and nifH mutant inoculated controls, Kp342 inoculation resulted in an increase in dry weight, chlorophyll content, total N, and N concentration in the plants. In addition, nitrogen deficiency symptoms were relieved and ¹⁵N was diluted in the plant tissue and in chlorophyll. Production of dinitrogenase reductase within the plant by Kp342 was also shown.

The subject invention also concerns nitrogen fixing endophytic bacteria isolated from a nitrogen efficient plant. The isolated bacteria can be utilized in the methods of the present invention. In one embodiment, the nitrogen fixing bacteria are resistant to a defense response of a plant. In a specific embodiment, the nitrogen fixing bacteria fail to express, and/or express lower levels of, one or more extracellular components, such as flagella or secretion systems. In one embodiment, the bacteria do not express, or express a mutant nonfunctional gene or gene product from, one or more of a sip, spa, or fli gene. In a specific embodiment, the sip gene is sipB, the spa gene is spaS, and the fli gene is fliC or fliB. In one embodiment, the bacteria are seed borne and can be transferred to the next crop of plants by their presence in the seed obtained from a plant colonized by the bacteria. In a specific embodiment, the bacteria is a Klebsiella pneumoniae. In an exemplified embodiment, the bacteria is Klebsiella Kp342.

Klebsiella pneumoniae cell cultures (designated as “Kp342) were deposited with American Type Culture Collection (ATCC), P.O. Box 1549, Manassas, Va. 20108, on Jun. 27, 2005. The subject cell cultures have been deposited under conditions that assure that access to the cultures will be available during the pendency of this patent application to one determined by the Commissioner of Patents and Trademarks to be entitled thereto under 37 CFR 1.14 and 35 U.S.C. 122. The deposit will be available as required by foreign patent laws in countries wherein counterparts of the subject application, or its progeny, are filed. However, it should be understood that the availability of a deposit does not constitute a license to practice the subject invention in derogation of patent rights granted by governmental action.

Further, the subject culture deposit will be stored and made available to the public in accord with the provisions of the Budapest Treaty for the Deposit of Microorganisms, i.e., it will be stored with all the care necessary to keep it viable and uncontaminated for a period of at least five years after the most recent request for the furnishing of a sample of the deposit, and in any case, for a period of at least thirty (30) years after the date of deposit or for the enforceable life of any patent which may issue disclosing the culture. The depositor acknowledges the duty to replace the deposit should the depository be unable to furnish a sample when requested, due to the condition of the deposit. All restrictions on the availability to the public of the subject culture deposit will be irrevocably removed upon the granting of a patent disclosing it.

The subject invention also concerns means to increase the number of free-living nitrogen-fixing bacteria in plants. Mutants of nitrogen-fixing endophytic bacteria can be generated that are resistant to plant defense responses. These mutants are generated by exposing the bacteria to extracts of tissue from plants whose defense responses have been induced. For example, bacteria are exposed to tissue extracts from a plant in which ethylene-mediated plant defense responses have been induced. Those bacteria that survive the exposure are selected and then examined to be certain that desirable phenotypes such as nitrogen fixation are maintained. The nitrogen fixing, defense response resistant mutants of the invention can colonize plants in much higher numbers than bacteria that have not been selected for resistance to plant defense responses. In one embodiment, the bacteria exposed to the tissue extracts are bacteria that do not express one or more extracellular components and/or that express lower levels of one or more extracellular components, such as flagella or secretion apparatus. In another embodiment, the bacteria do not express or express one or more mutant non-functional sip, spa, or fli genes. In a specific embodiment, the sip gene is sipB, the spa gene is spaS, and the fli gene is fliC or fliB. Preferably, the mutant bacteria are resistant to SA-independent plant defense responses. The higher number of cells colonizing the plant can provide enough fixed N to relieve the nitrogen deficiency symptoms of a nitrogen starved plant. In one embodiment, the plant is a non-leguminous plant, such as an agronomically important grass, e.g., wheat, rice, maize, barley, oats, sorghum, and rye. In a specific embodiment, the plant is a wheat plant. In a further embodiment, the plant is selected or produced that has decreased defense responses to bacteria. For example, the plant can express a mutant npr1 gene.

In one embodiment, a mutant bacterial strain of the invention that is resistant to plant defense responses is used as an inoculant for any crop that requires nitrogen fertilizer. Bacterial strains of the invention can be selected that have a broad host range and can be used to inoculate and colonize any plant. Mutant bacteria of the invention that have a narrower plant host range can also be used. Bacterial strains contemplated within the scope of the invention include those that are typically poor soil saprophytes and, thus, plants may require annual inoculation. In one embodiment, the bacterial inoculant only needs to be applied at the time of planting compared to untreated non-leguminous plants where at least two applications of nitrogen fertilizer is required. The bacterial inoculant can provide a constant source of fixed N whereas the availability of the nitrogen of fertilizer is based on the time of application and the amount of leaching that occurs in the soil. The mutant bacteria can be inoculated onto any part of a plant, including seeds, roots, and leaves.

The subject invention also concerns methods for increasing total N of a plant. In one embodiment, the plant, plant tissue, or a plant seed is inoculated with an effective amount of bacteria capable of fixing nitrogen and then the plant or the seed is grown. In another embodiment, the bacteria of the invention are seed borne and thus are present in seed obtained from a plant already colonized by the bacteria. Thus, seed from a plant colonized by the nitrogen fixing bacteria can be grown to produce a plant that is itself colonized with the bacteria, thereby avoiding the process of inoculating the seed or plant at the time of planting or after planting. In one embodiment, the nitrogen fixing bacteria fail to express, and/or express lower levels of, one or more extracellular components, such as flagella or secretion systems. In one embodiment, the bacteria do not express, or express a mutant nonfunctional gene or gene product from, one or more of a sip, spa, or fli gene. In a specific embodiment, the sip gene is sipB, the spa gene is spaS, and the fli gene is fliC or fliB. In an exemplified embodiment, the bacteria is Klebsiella Kp342. In one embodiment, the plant is a non-leguminous plant, such as an agronomically important grass, e.g., wheat, rice, maize, barley, oats, sorghum, and rye. Any bacterium that can colonize a plant and that can fix nitrogen or that can be genetically engineered to fix nitrogen, e.g., by transformation with nif polynucleotide(s) (see, for example, Genbank accession no. X 13303 (SEQ ID NO: 10)), is contemplated within the scope of the present invention. Methods and materials for transforming a bacterium with a polynucleotide which is expressed in the bacterium are known in the art. Bacteria that can be used in the subject invention include, but are not limited to, Klebsiella sp., Enterobacter sp., Pantoea sp., Agrobacterium sp., Alcaligenes sp., Azorhizobium sp., Ayospirillium sp., and Pseudomonas sp. In one embodiment, the bacteria is a Klebsiella sp. In an exemplified embodiment, the bacteria is Klebsiella pneumoniae and the strain is Kp342. The progeny and derivatives of any bacteria of the invention are also contemplated within the scope of the invention.

The subject invention also concerns materials and methods for eliminating or decreasing the number of bacterial pathogens residing within plant tissue. In one embodiment, plant defense responses to one or more bacterial pathogens are induced in the plant. The plant defense response can be, for example, an ethylene-mediated defense response. The plant can be treated, for example, with a chemical that induces a defensive response. The plant can also be prepared wherein the plant expresses or overexpresses a gene, such as an NPR1 gene (see, for example, Genbank accession nos. NM 105102; AF527176 (SEQ ID NOs: 3 and 4); NM 191394; AF480488 (SEQ ID NOs: 5 and 6); AX041006 and WO 00/065037 (SEQ ID NOs: 7, 8, and 9), which confers disease resistance in plants. In one embodiment, the bacterial pathogen is Salmonella sp.

The subject invention also concerns materials and methods for expressing or inducing defense responses, such as ethylene-mediated responses, in a plant in order to reduce the number of pathogenic bacteria that colonize the plant. The plant can be treated such that plant defense responses are expressed or induced in the plant. In one embodiment, a plant is engineered to express or overexpress an NPR1 gene. The defense responses reduce the number of bacteria colonizing the plant or prevent the plant from being colonized by a large number of bacteria. In one embodiment, the bacterial pathogen is Salmonella sp.

The subject invention also concerns plants that exhibit enhanced nitrogen fixation and/or that are resistant to colonization by a bacterial pathogen. In one embodiment, a plant can be prepared by inoculating the plant or plant tissue with an effective amount of nitrogen fixing bacteria of the invention. In one embodiment, plant parts, such as roots, are inoculated with bacteria for colonization of the plant. In an exemplified embodiment, a plant is prepared by inoculating plant seeds with nitrogen fixing bacteria of the invention and growing a plant from the seed. In another embodiment, the bacteria of the invention are seed borne and thus are present in the seed obtained from a plant already colonized by the bacteria. Thus, seed from a plant colonized by the nitrogen fixing bacteria can be grown to produce a plant that is itself colonized with the bacteria, thereby avoiding the process of inoculating the seed or plant at the time of planting or after planting. In one embodiment, plants are engineered to express defense responses. In another embodiment, plants are engineered wherein defense responses can be induced upon exposure of the plant to a substance or condition. The defense response can be, for example, an ethylene-mediated defense response. In one embodiment, the defense responses can be salicylic acid-mediated (SA-mediated) or salicylic acid-independent (SA-independent) responses. In a specific embodiment, a plant is engineered to express or overexpress an NPR1 gene. In a specific embodiment, the plant is resistant to Salmonella sp.

Plants within the scope of all methods and materials of the present invention include monocotyledonous plants, such as rice, wheat, barley, oat, sorghum, maize, rye, sugarcane, pineapple, onion, banana, coconut, lily, grass, and millet; and dicotyledonous plants, such as, for example, peas, alfalfa, tomato, tomatillo, melon, chickpea, chicory, clover, kale, lentil, soybean, tobacco, potato, sweet potato, radish, cabbage, rape, apple trees, grape, cotton, sunflower, thale cress, canola, citrus (including orange, mandarin, kumquat, lemon, lime, grapefruit, tangerine, tangelo, citron, and pomelo), pepper, bean, and lettuce. Plants within the scope of the present invention also include conifers.

Techniques for transforming plant cells with a gene are known in the art and include, for example, Agrobacterium infection, biolistic methods, electroporation, calcium chloride treatment, etc. Transformed cells can be selected, redifferentiated, and grown into plants using standard methods known in the art. The progeny of any transformed plant cells or plants are also included within the scope of the present invention.

All patents, patent applications, publications, and information associated with accession numbers referred to or cited herein are incorporated by reference in their entirety, including all figures, tables, and sequences, to the extent they are not inconsistent with the explicit teachings of this specification.

### MATERIALS AND METHODS FOR EXAMPLES 1-4

Kp342 (Chelius and Triplett 2000) and a nifH mutant of Kp342 were grown overnight on Luria-Bertani agar plates at 28° C. DNA:DNA hybridization assays have classified Kp342 as a member of K. pneumoniae (Dong et al, 2003a). The four treatments in each experiment included uninoculated plants and plants inoculated with Kp342, the nifH mutant of Kp342, and dead cells of Kp342. Prior to inoculation, Kp342 and Kp342 nifH mutant cells were re-suspended in phosphate-buffered saline creating a thick cell suspension containing 5×10⁹ CFU/ml. For dead cells, Kp342 was cultured and re-suspended as described above, but autoclaved for 30 min. The heat killed cell suspension was allowed to reach room temperature before it was applied to wheat seeds. Cell death was confirmed by failure to grow on LB.

The Kp342 nifH mutant was constructed as follows. Primers nifH1f (5′-GCCTGCAGATGACCATGCGTCAATGCGCC-3′) (SEQ ID NO: 1) and nifH876r (5′-GCGAATTCCGCGTTTTCTTCGGCGGCGGT-3′) (SEQ ID NO: 2) based on the nifH sequence of K. oxytoca M5al (formerly K. pneumoniae M5al, Suarez et al., 1995) were used with 100 ng of Kp342 DNA in PCR using the conditions described previously (Chelius and Triplett 2000). The PCR product was purified with a Qiagen PCR purification kit and then ligated to pGEM-T Easy vector. A 1.7 kb fragment containing nifH gene and part of nifD was excised from pSA30 by double digestion with EcoRI and BamHI. The nifHDKY operon from K. pneumoniae is present in pSA30 (Cannon et al., 1979). This fragment was inserted into EcoRI/BamHI digested vector pUC18, resulting in plasmid pH1. A 1.4 kb fragment from pKRP11 (Reece and Phillips 1995) containing nptII downstream of a constitutive promoter was excised with HindIII and blunted with Klenow. Following BglII digestion of pH1 and subsequent blunting, pI1 was created by inserting the fragment from pKRP11 into the BglII site of pH1. To exchange the inserted nifH for the wild type allele on the chromosome, the 3.1 kb fragment containing nifHD′-Km was excised from pI1 by digestion with EcoRI and PstI. This fragment was blunted and ligated into the PstI/SmaI digested plasmid pJQ200KS+ followed by marker exchange (Scupham and Triplett 1997). Nif isolates were then selected on an N-free medium with ampicillin and kanamycin. Marker exchange was confirmed by southern hybridization with nptII in isolates with no acetylene reduction activity.

The soil mixtures for each experiment were perlite and vermiculite each mixed with sand in a 1:1 ratio by volume. Once mixed the two soil mixtures were autoclaved at 121° C. for 2 hours, allowed to cool overnight, and autoclaved again for 2 hours. The soil was allowed to cool to room temperature before adding 10 mg of ¹⁵NH₄NO₃ (11.7 atom % ¹⁵N excess) per Kg wet weight of soil. To ensure the proper distribution of ¹⁵N, the soil was mixed thoroughly twice daily for 2 weeks prior to planting. Finally, 2 L pots were filled with about 2.5 kg of the ¹⁵N-labeled soil mixture.

Seeds of Triticuim aestivum L. cv. Trenton (a commercial cultivar) were surface sterilized as described previously (Chelius and Triplett 2000). After the surface sterilization, seeds were submerged in the appropriate inoculum suspension described above at room temperature for about two hours and then five seeds per pot were placed in the soil mixtures. The remainder of the cell suspension was applied in equal amounts on top of the planted seeds. After plants emerged, they were thinned to two plants per pot. There were 10 replicates per treatment. To measure chlorophyll, a Minolta SPAD 502 meter was used. Relative chlorophyll concentration is unitless and is a ratio of transmittance between red (650 nm) and infrared (940 nm) emissions through the leaf.

Plants were grown under greenhouse conditions, with 10 hours nights at 21° C. (±2° C.) and 14 hours days at 23° C. (±2° C.). Artificial light ensured a minimum light level of 120 μeinsteins/m²/sec. Plants were watered as needed with a nutrient solution containing (in μM): 5 CaCl₂, 1.25 MgSO₄, 5 KCl, 1 KH₂PO₄, 0.162 FeSO₄; and micronutrients (in nM): 2.91 H₃BO₃, 1.14 MnSO₄, 0.76 ZnSO₄, 0.13 NaMoO₄, 0.14 NiCl₂, 0.013 CoCl₂, and 0.19 CuSO₄. Six weeks after planting, plants were washed to remove the attached soil mix. Roots and shoots were separated and dried at 65° C. for 48 hours and ground through a 0.5 mm mesh. Ten mg of roots and shoots were assayed for ¹⁵N content by mass spectrometry. Using these data, the % N in plant tissue derived from the atmosphere was estimated from ¹⁵N tissue analysis of roots and shoots. Chlorophyll ¹⁵N content was determined by mass spectrometry after acidification to pheophytin (Kahn et al., 2002).

Sand-perlite and sand-vermiculite sub-samples (6 of each) and seeds were tested for total N content by Kjeldahl analysis. The extent of endophytic colonization, inoculum preparation, planting, in planta NifH visualization, statistics, and harvesting were done as described previously (Chelius and Triplett 2000; Dong et al., 2003a, 2003b, 2003c).

Following are examples which illustrate procedures for practicing the invention. These examples should not be construed as limiting. All percentages are by weight and all solvent mixture proportions are by volume unless otherwise noted.

### EXAMPLE 1

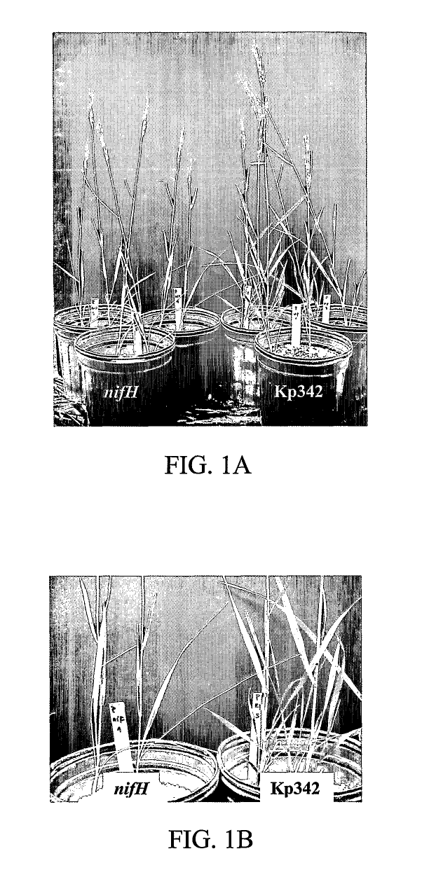

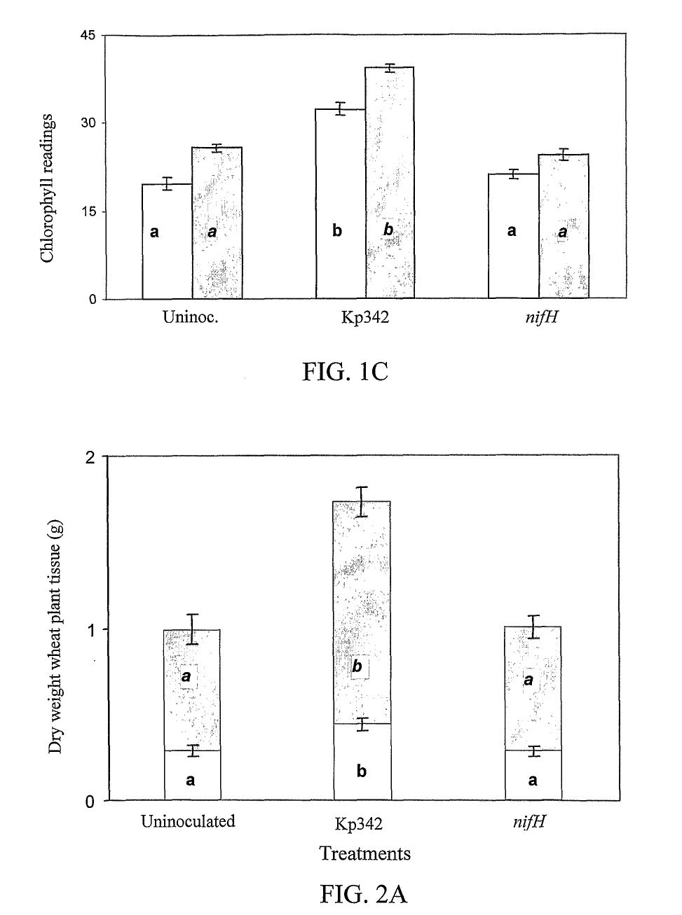

After six weeks of growth in the greenhouse without nitrogen fertilizer, uninoculated plants and plants inoculated with the nifH mutant were stunted and chlorotic showing severe signs of nitrogen deficiency (FIGS. 1A and 1B). Only wheat plants inoculated with Kp324 appeared taller, more robust, and greener than the controls regardless of the medium in which they were grown (FIGS. 1A and 1B). Two plant culture media (1:1 sand-perlite and 1:1 sand-vermiculite) were used to illustrate the reproducibility of the results. The results of dead cell inoculum treatment for all parameters measured were not statistically different from the nifH or uninoculated treatments (data not shown). Chlorophyll levels in Kp342-inoculated plants were significantly higher than chlorophyll levels found in control plants (FIG. 1C).

### EXAMPLE 2

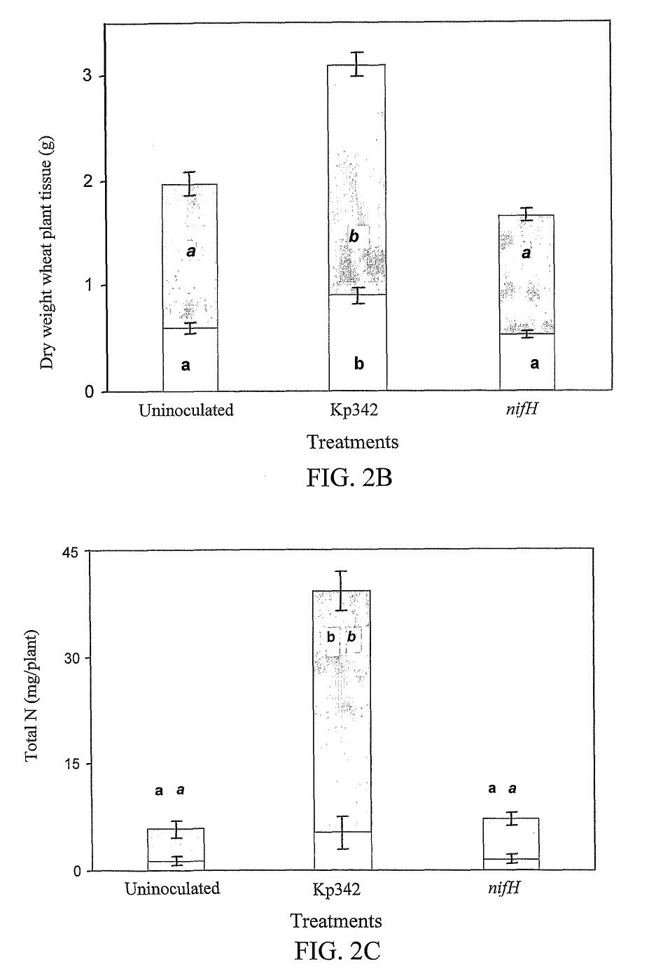

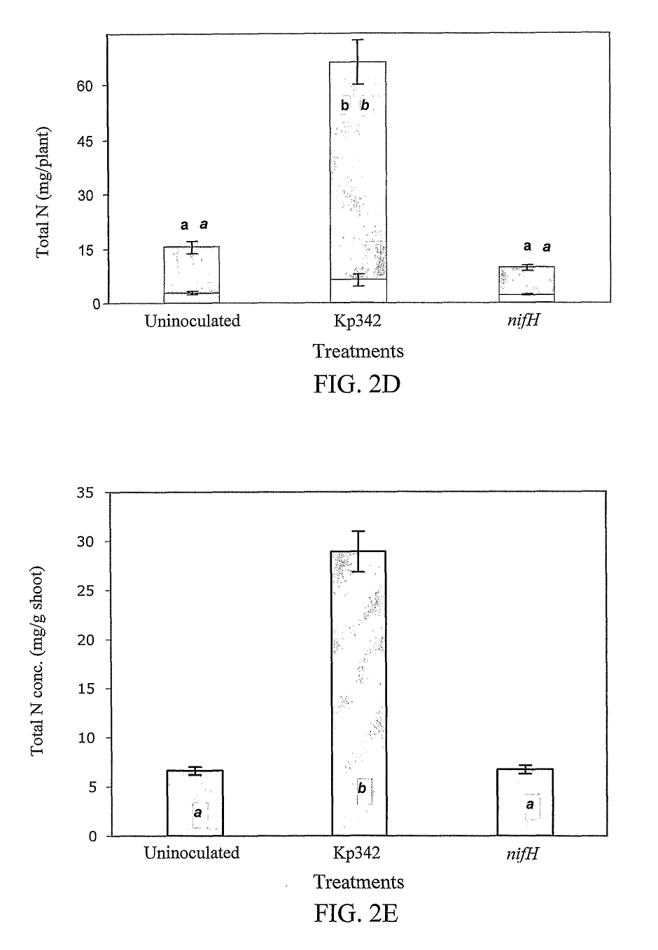

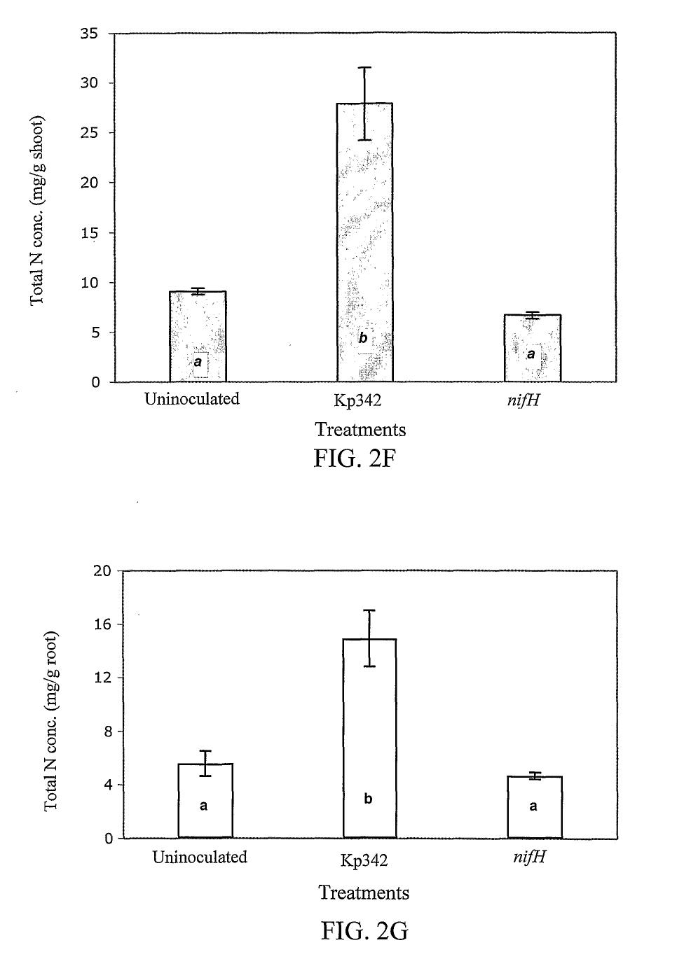

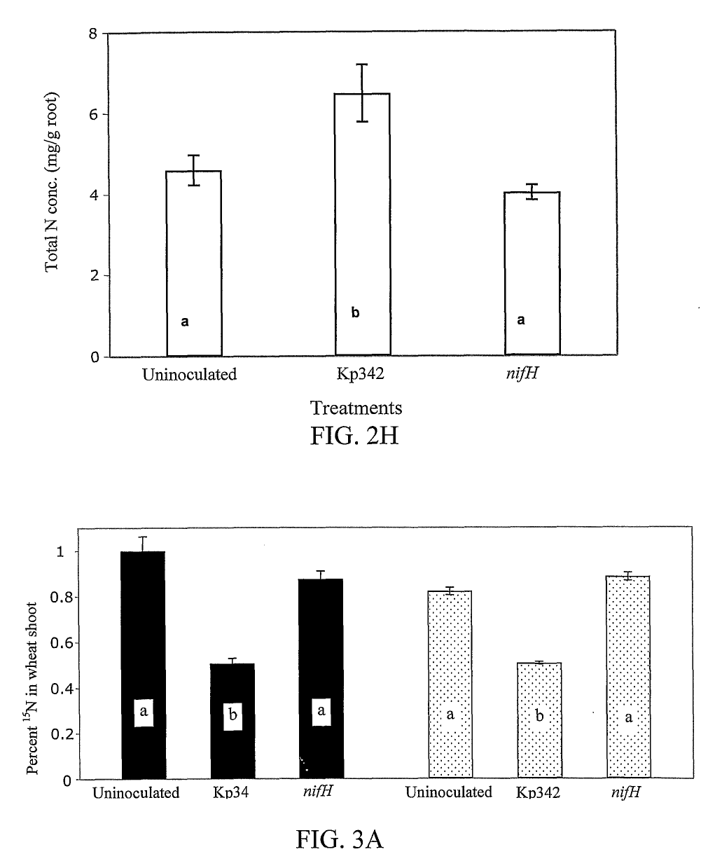

Kp342 also significantly increased the dry weight of roots and shoots compared to controls regardless of the growth medium (FIGS. 2A and 2B). Roots and shoots of Kp342-inoculated plants were always at least 50% larger in dry weight compared to the untreated controls. Changes in total N per plant with Kp342 inoculation were even more dramatic. In sand-perlite, the percent increase in total N for Kp342 inoculated plants grown was 244 and 498% greater for roots and shoots, respectively, compared to the nifH control (FIG. 2C and D). Compared to the uninoculated control, Kp342 accumulated 285 and 654% more total N in shoots and roots, respectively. In sand-vermiculite Kp342 inoculated plants had 180 and 707% more total N compared to the nifH inoculated plants in the roots and shoots, respectively. In the same growth medium, the total N of Kp342-inoculated plants increased 120 and 378% respectively for roots and shoots compared to uninoculated plants (FIGS. 2C and 2D).

The concentration of N in plant tissues also increased significantly with Kp342 inoculation compared to the controls. In sand-perlite, the percent increase in total N concentration for Kp342 inoculated plants grown was 318 and 368% greater for roots and shoots, respectively, compared to the nifH control (FIGS. 2E and 2G). Compared to the uninoculated control, Kp342 accumulated an N concentration 317 and 394% higher in shoots and roots, respectively (FIGS. 2E and 2G). In sand-vermiculite, Kp342 inoculated plants had an N concentration 161 and 381% higher than the nifH inoculated plants in the roots and shoots, respectively (FIGS. 2F and 2H). In the same growth medium, the N concentration of Kp342-inoculated plants increased 120 and 378%, respectively for roots and shoots compared to uninoculated plants (FIGS. 2F and 2H).

### EXAMPLE 3

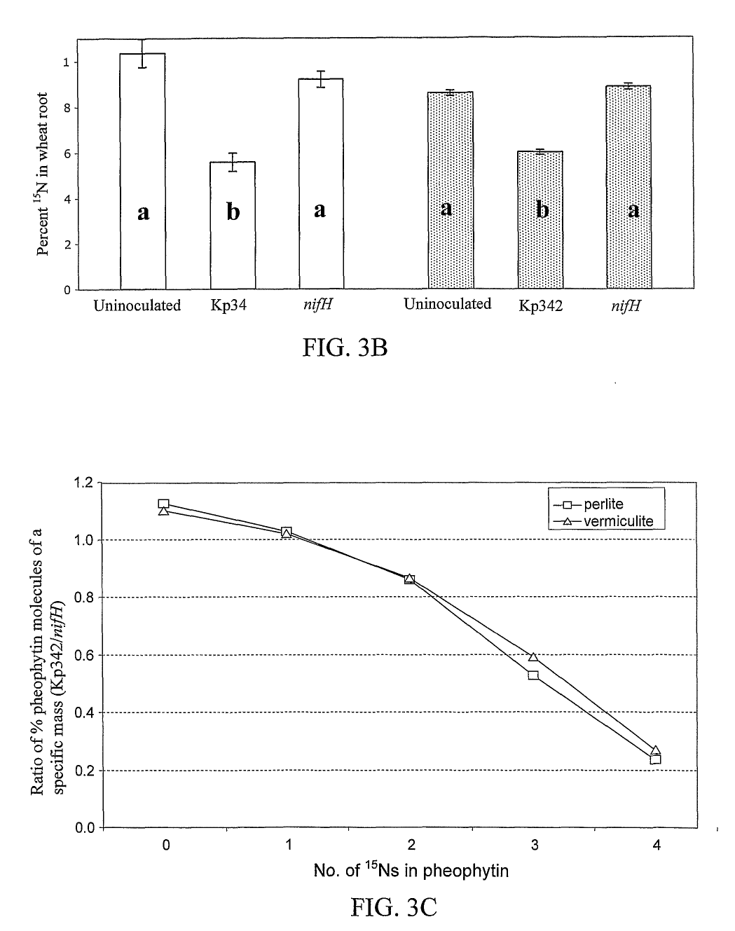

To verify that much of the N in these plants was derived from the atmosphere, the plant growth media were evenly labeled with 10 mg of 11.7 atom percent excess ¹⁵NH₄NO₃ per kg of sand-vermiculite and sand-perlite mixes. The ¹⁵N concentration of Kp342 inoculated plants was significantly lower than in the controls as a result of nitrogen fixation (FIG. 3). As the primary source of ¹⁵N in the plants is from the enriched ¹⁵N in the soil, the extent of the dilution of the ¹⁵N isotope can be used to calculate the amount of N in the plants derived from the atmosphere. This can be calculated by % NF=(1−A/B)×100, where % NF=the percent of N in the nitrogen-fixing system derived from the atmosphere; A=% ¹⁵N in the nitrogen fixing system; and B=% ¹⁵N in the non-fixing system (Boddey et al., 1983). When the comparison is made with the nifH control, the Kp342-inoculated plants received 42% and 41% of their nitrogen from N₂ for plants grown in sand-perlite and sand-vermiculite, respectively. When the comparison is made with the uninoculated control, the Kp342-inoculated plants received 49% and 37% of their nitrogen from N₂ when the plants were cultured in sand-perlite and sand-vermiculite, respectively.

The remaining N in Kp342-inoculated plants came primarily from the plant growth media since the N content of seeds was very low, being less than 0.006% of the total N in the pots at the time of planting. This was calculated by determining the amount of N in three sets of 10 seeds taken from the same bag of seeds, the amount of N as ¹⁵NH₄NO₃ added to the soil mixes, and the total amount of N in the soil mixes. On average, the sand-vermiculite and sand-perlite pots contained 91.6 and 74.6 mg of N, respectively, at the start of the experiment. This includes an average of 8.0 and 6.8 mg of 11.7 atom % excess ¹⁵NH₄NO₃ in sand-vermiculite and sand-perlite, respectively. However, Kp342-inoculated plants contained 132.3 and 78.2 mg N per pot (2 plants/pot) in sand-vermiculite and sand-perlite, respectively. That is, the plants contained statistically significantly more N (44% and 5% more N in sand-vermiculite and sand-perlite cultured plants, respectively) than was present in the entire pot (including seed N) at the start of the experiment. In contrast, the nifH mutant-inoculated plants contained only 27.9 and 12.8 mg N per pot (2 plants per pot), respectively for the sand-vermiculite and sand-perlite experiments. Thus, the nifH mutant-inoculated plants contained far less N than was present in the pots (including seed N) at the beginning of the experiment. The nutrient solution contained no detectable N throughout the experiment with a limit of detection of 0.3 ppm. A concentration of 0.3 ppm N in the nutrient solution is insufficient to relieve the nitrogen deficiency symptoms observed here in the uninoculated plants or plants inoculated with the nifH mutant of Kp342.

Assuming that the % N in the plants derived from the atmosphere is that calculated based on the ¹⁵N abundance of nifH mutant- and Kp342-inoculated plants, the Kp342-inoculated plants were capable of mining about 62 and 86% of their total N from the growth medium, respectively for the sand-perlite and sand-vermiculite mixtures. That amount combined with the amount of N₂ fixed from the atmosphere allowed for vigorous plant growth and relieved the nitrogen deficiency symptoms. Thus, the increased availability of N to the Kp342-inoculated plants permitted more root growth allowing these plants to absorb a majority of the N present in the soil. In contrast, the nitrogen-limited control plants had very small roots that were only able to absorb 19 and 21% of the N from the growth medium, respectively for the sand-perlite and sand-vermiculite mixtures.

Fixed N was also incorporated into chlorophyll. Chlorophyll was extracted from the plant tissue and acidified to pheophytin. The proportion of ¹⁵N/¹⁴N in the four N atoms of pheophytin was determined by mass spectrometry. A pheophytin molecule from the nifH treatment was more than twice as likely to be fully labeled with ¹⁵N than in the Kp342 treatment regardless of the growth medium used for plant culture. Similarly, a significantly higher proportion of pheophytin molecules were labeled with two or three ¹⁵N atoms in the nifH treatment compared to the Kp342 treatment. Thus, just as nitrogen fixation in Kp342-inoculated plants diluted the ¹⁵N label in total plant tissue, this dilution was also observed directly in a plant product, chlorophyll. The mean mass of pheophytin was 872.454 (±0.041), 872.234 (±0.0036), 872.398 (±0.027), and 872.238 (±0.0031) for the nifH and Kp342 treatments in sand-perlite, and the nifH and Kp342 treatments in sand-vermiculite, respectively. The mass of pheophytin with all four N atoms as ¹⁴N is 871.6. The decline in average pheophytin mass with the Kp342 treatment compared to the nifH control was statistically significant at the 1% level of confidence in both plant growth media.

### EXAMPLE 4

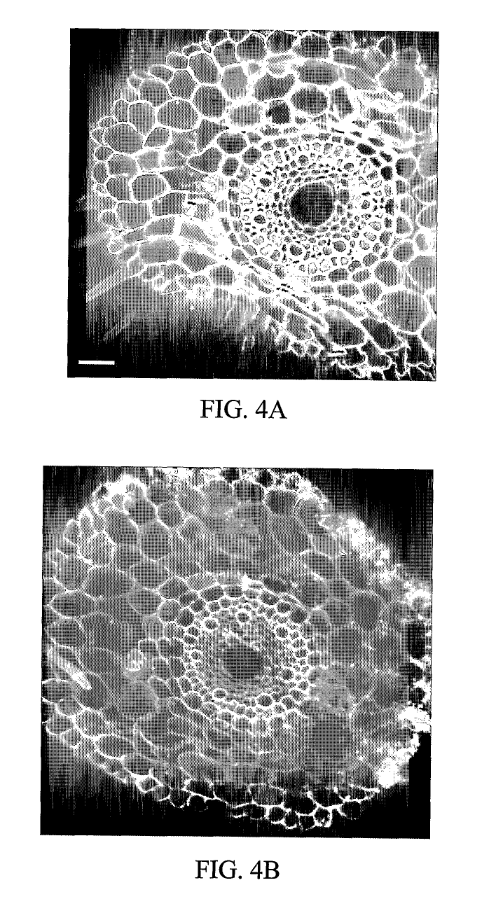

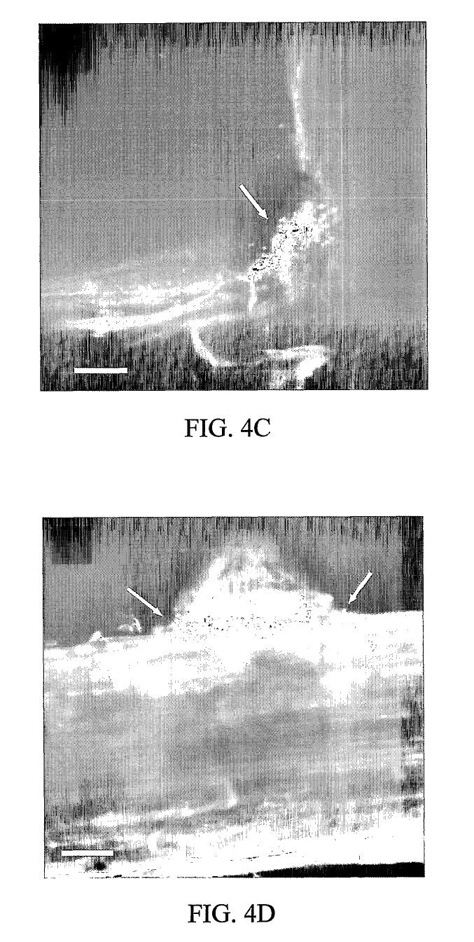

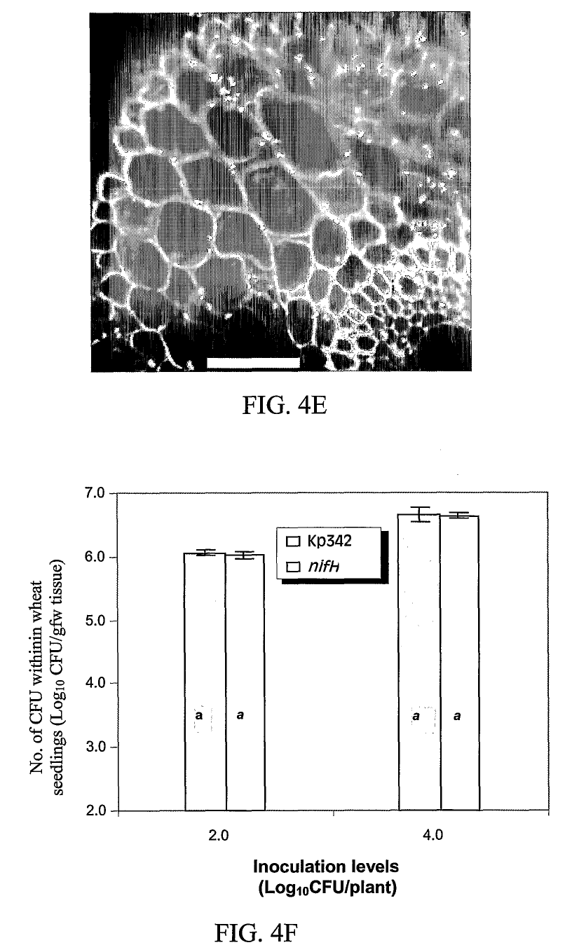

Kp342 was present within the roots of plants and were producing dinitrogenase reductase in planta (FIGS. 4A-4F). The concentrations of Kp342 and nifH mutant cells in the roots were identical regardless of the number of cells in the inoculum (FIG. 4F). Confocal images of root cross-sections and around lateral root emergence showed similar colonization patterns and abundance by both strains (FIGS. 4A-4E). Thus, the lack of nutritional benefit from the nifH cells was not caused by a failure of the mutant to colonize the exterior or interior of roots. Dinitrogenase reductase production by GFP (green fluorescent protein)-labeled Kp342 cells in roots was determined by scanning confocal laser microscopy (FIG. 4E). As done previously in maize (Chelius and Triplett 2000), the co-localization of both fluorophores (green for GFP and red for NifH) renders a yellow color allowing the simultaneous localization of wild type Kp342 expressing NifH. NifH expression by Kp342 was observed in several areas of the roots including cross sections (FIG. 4E). Nitrogen deficiency symptoms were not relieved in cultivars Russ or Stoa with Kp342 inoculation but biomass did increase significantly in Stoa (Table 1).

TABLE 1
Comparison of three wheat cultivars for their ability
to enhance growth in the greenhouse and relieve nitrogen
deficiency symptoms upon inoculation with Kp342.

| | dry weight | | Chlorophyll | |
|---|---|---|---|---|
| | (μg shoots/plant) ± s.e. | | (units/unit leaf area) ± s.e. | |
| Cultivar | Kp342 | uninoculated | Kp342 | uninoculated |
| Russ | 416 ± 3 | 402 ± 2 | 26.2 ± 1.2 | 25.3 ± 1.4 |
| Stoa | 380 ± 4 | 269 ± 1 | 26.8 ± 3.7 | 26.1 ± 0.5 |
| Trenton | 790 ± 13 | 257 ± 4 | 35.6 ± 3.4 | 22.9 ± 3.9 |

Measurements were taken after six weeks of growth in the absence of nitrogen fertilizer in a sand-vermiculite mixture. Biomass is measured as the dry weight of shoots per plant. Nitrogen deficiency measured by assaying the amount of chlorophyll in arbitrary units per unit area in the leaves.
(s.e. = standard error about the mean)

### MATERIALS AND METHODS FOR EXAMPLES 5-8

Bacterial strains and inoculum preparation. The bacterial strains used in Examples 5-8 are listed in Table 2. BA3104 was constructed by sequential P22 transduction into 14028 using a P22HTint lysate grown on the SL3201 fliC::Tn10 fliB::MudJ strain kindly provided by Dr. Allison O'Brien (Schmitt et al., 2001). pHC112 was constructed by amplifying the spaS gene of 14028 (nucleotides 28 to 1327 of GenBank accession number AE008832) using Taq DNA polymerase. The spaS fragment was cloned into pCR-2.1-TOPO (Invitrogen), removed using EcoRI, and cloned into the EcoRI site of pWSK29 (Wang and Kushner 1991). pHC113 was constructed in the same way except that the sipB gene was amplified (nucleotides 18133 to 20138 of GenBank accession number AE008831). Bacterial strains were cultured and inoculum prepared as described previously (Dong et al., 2003a; Dong et al., 2003b) with the exception of the experiments designed to estimate the number of infection events. For these experiments the inoculum strains were composed of a mixture of either 14028 or Kp342 with and without a constitutively expressed GFP gene.

TABLE 2
Bacterial strains used herein.

| Strains | Abbreviations | Comments | Reference |
|---|---|---|---|
| K. pneumoniae 342 | Kp342 | maize endophyte | (Chelius and |
| | | K. pneumoniae 342 | Triplett 2000) |
| S. enterica serovar | 14028 | Type strain provided by | American |
| Typhimurium ATCC | | American Type Culture | Type Culture |
| 14028 | | Collection | Collection |
| BA1502 (14028 | spaS | TTSS SPI1 structural mutant | (Ahmer et al., |
| spaS1502::MudJ) | | | 1999) |
| BA1502/pHC112 | spaS | spaS mutant complemented | Present |
| | complement | with the spaS gene | application |
| BA1577 (14028 | sipB | TTSS SPI1 structural mutant | (Ahmer et al., |
| sipB1577:MudJ) | | | 1999) |
| BA1577/pHC113 | sipB | sipB mutant complemented with | Present |
| | complement | the sipB gene | application |
| BA3104 (14028 | fliC/fljB | lacks two flagellin biosynthetic | Present |
| fliC::Tn10 fljB::MudJ | | genes | application |
| P. syringae | N/A | Contains avrRpt2 on plasmid | (Kunkel et al., |
| DC3000avrRpt2 | | PV288. Provided by Andrew | 1993) |
| | | Bent University of Wisconsin- | |
| | | Madison | |

Scanning Confocal Laser Microscopy (SCLM). The methodology used here for SCLM was previously described (Dong et al., 2003a; Dong et al., 2003b). Using this methodology, hypocotyls of Medicago truncatula mutant sickle (ski) and Medicago truncatula Jermalong were observed under SCLM with 20× magnifications through z-sections ranging from 0.5 to 2 μm in thickness.

Seed surface sterilization, germination, inoculation, plant culture and harvest. The plants used in this work are listed in Table 3. The manipulation of plants, from seed surface sterilization to plant harvest, were carried out by methods developed previously (Dong et al., 2003a; Dong et al., 2003b).

TABLE 3
Plants used herein.

| Plant line | Comment | Reference |
|---|---|---|
| Medicago sativa cv. | Common line for alfalfa sprout | |
| CUS101 | production | |
| Medicago truncatula | Provided by Doug Cook, Univ. of | |
| Gaerten cv. A17 | California, Davis | |
| Medicago truncatula | Provided by Doug Cook, Univ. of | (Penmetsa and |
| mutant sickle (skl) | California, Davis | Cook, 1997) |
| Medicago truncatula | Provided by Barry Rolfe, Australian | |
| Jester | National University | |
| Medicago truncatula | Provided by Edwin Bingham, | |
| Jermalong | University of Wisconsin-Madison | |
| Arabidopsis thaliana cv. | ABRC | |
| Col-0 | | |
| Arabidopsis thaliana cv. | Provided by Julie Stone University of | (Cao et al., 1994) |
| Col-0 PR1::GUS | Nebraska-Lincoln | |
| Arabidopsis thaliana cv. | Provided by Julie Stone University of | (Reuber et al., 1998) |
| Col-0 nahG | Nebraska-Lincoln | |
| Arabidopsis thaliana cv. | Provided by Julie Stone University of | (Cao et al., 1994) |
| Col-0 npr1-4 | Nebraska-Lincoln | |
| Triticum aestivum cv. | hard red spring wheat line developed at | |
| Trenton | North Dakota State Univ. in 1995 | |

Determination of microbial population within surface sterilized plant tissue. With the exception of the M. truncatula sickle experiment, where the whole plant tissue was used to determine microbial populations, only the root and hypocotyl were examined for bacterial colonization. The procedures used for surface sterilization, determination of endophytic microbial populations and statistical analysis were done as described previously (Dong et al., 2003a; Dong et al., 2003b).

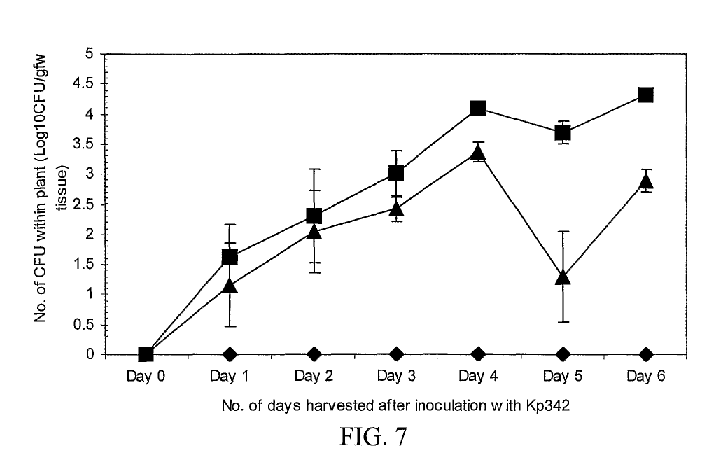

Assurance of endophytic colonization results. To ensure the endophytic colonization numbers presented reflect only the number of cells within the interior of plant tissue, previously developed methods were followed (Dong et al., 2003a; Dong et al., 2003b). Furthermore, day 0 of the time course experiment (FIG. 7), serves as a control to ensure that the endophytes do not enter the plants through wounds caused during harvesting or through the root surface as a result of the surface sterilization procedure. Day 0 data show that no Kp342 cells were recovered from the interior of alfalfa seedlings within one hour after inoculation. This suggests that the methods used here to estimate microbial population within plants do not contribute to endophytic invasion of the apoplast.

Induction of ethylene response in seedlings. To induce ethylene responses, seedlings were cultured in growth medium as described previously (Dong et al., 2003a) supplemented with 5 μM 1-aminocyclopropane-1-carboxylic acid (ACC). ACC was dissolved in water and filter sterilized prior to its addition to autoclaved plant growth media. In most experiments, seedlings were exposed to media containing ACC for 12 hours prior to inoculation.

In the ethylene time course experiments, gaseous ethylene was added to the plants cultured in closed tubes to a final concentration of 5 μM. The stopper on these tubes was removed each day, flushed with fresh air, stopped, and re-treated with sufficient ethylene to bring to a final concentration of 5 μM.

Preparation and use of 1-methylcyclopropene (1-MCP). The gaseous ethylene action inhibitor, 1-MCP, was prepared and stored as described by Hall et al. (2000). 1-MCP was generated from ETHYLBLOC, which was provided by A. B. Bleecker (University of Wisconsin—Madison). The concentration of 1-MCP in ETHYLBLOC is of 0.14%. A stock of 1-MCP of 100 ppm was created in a serum bottle of 121.5 ml in volume. This was accomplished by adding 19.44 mg of ETHYLBLOC and 0.5 ml of hot H₂O to the serum bottle and set to rest for 15 minutes. The stock was used to dispense 0.3 ml of headspace gas to 30 ml stopped test tubes where the plants were cultured, resulting in a final concentration of 1 ppm per tube. The plant cultures were placed under conditions as described previously (Dong et al., 2003a; Dong et al., 2003b) with the exception of a rubber stopper used to conceal 1-MCP. The stoppers were removed daily, flushed with air, stopped again and finally freshly prepared 1-MCP was added to the desired final concentration.

GUS histochemical staining and GUS fluorogenic assay. Roots of transgenic Arabidopsis thaliana Col-0 harboring a pathogenesis-related 1 (PR1) gene promoter fused to the bacterial uidA (β-glucuronidase) reporter gene (PR1::GUS) were inoculated with 10⁷ CFU S. enterica 14028, the 14028 sipB mutant, and the complemented sipB mutant,. Exogenous application of salicylic acid (5 mM) and infiltration of leaves with 10⁷ CFU of an avirulent strain of P. syringae DC3000 carrying the avrRpt2 on plasmid PV288 were used as positive controls (Kunkel et al., 1993; Ton et al., 2002). The histochemical assay was performed as described by Sundaresan et al. (1995) with slight modifications (Sundaresan et al., 1995). Plants were immersed in staining buffer (50 mM sodium phosphate pH7, 10 mM EDTA, 0.1% Triton X-100, 100 μg/ml chloramphenicol, 5 mM potassium ferricyanide and 0.5 mg/ml 5-bromo-4-chloro-3-indolyl-β-D-glucuronide (X-Glc). Plants were then vacuum infiltrated, incubated overnight at 37° C., and destained with 70% ethanol.

To conduct the quantitative GUS fluorogenic assay, whole plants were flash-frozen in liquid N₂ and crushed. The fluorogenic assay and protein extraction were done as described by (Jefferson et al, 1987). Protein concentration of the samples was determined using a BCA protein assay kit (PIERCE, Rockford, Ill.).

Following are examples which illustrate procedures for practicing the invention. These examples should not be construed as limiting. All percentages are by weight and all solvent mixture proportions are by volume unless otherwise noted.

### EXAMPLE 5

### Ethylene, a Signal Molecule for Induced Systemic Resistance in Plants, Decreases Endophytic Colonization

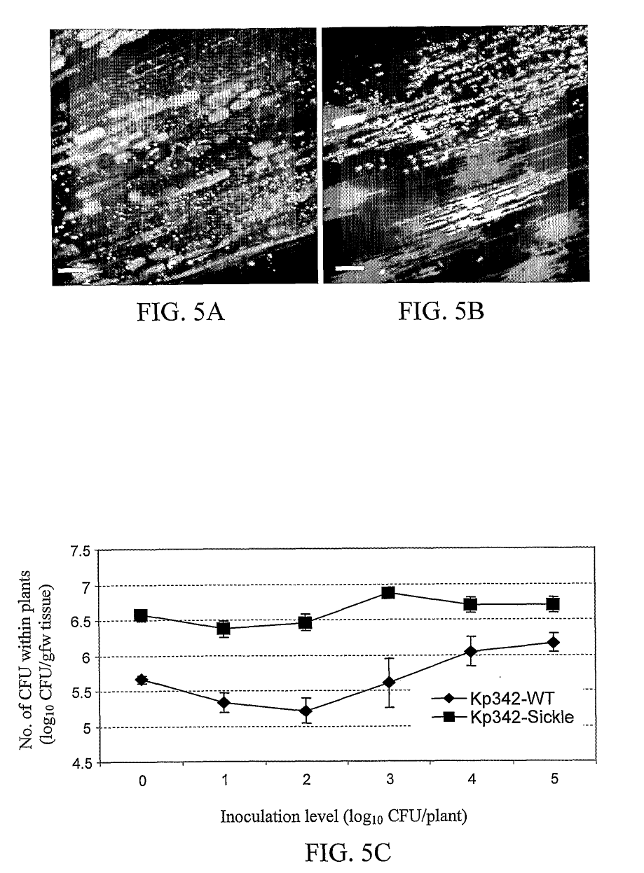

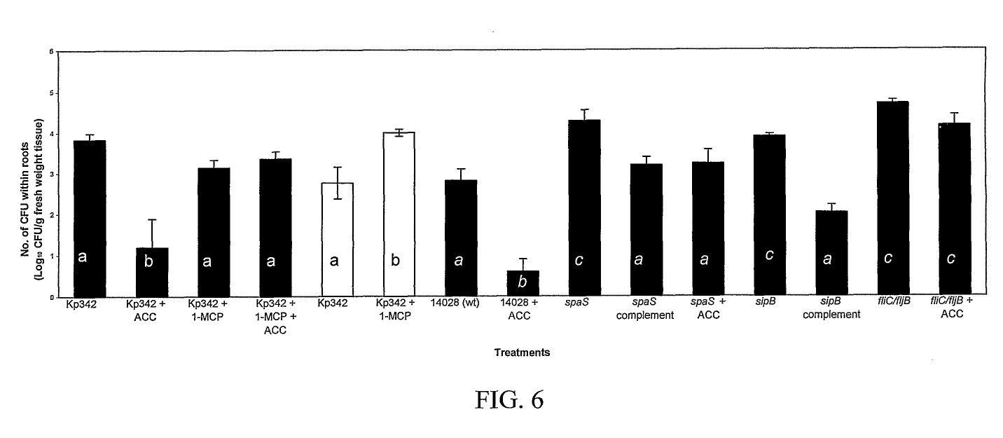

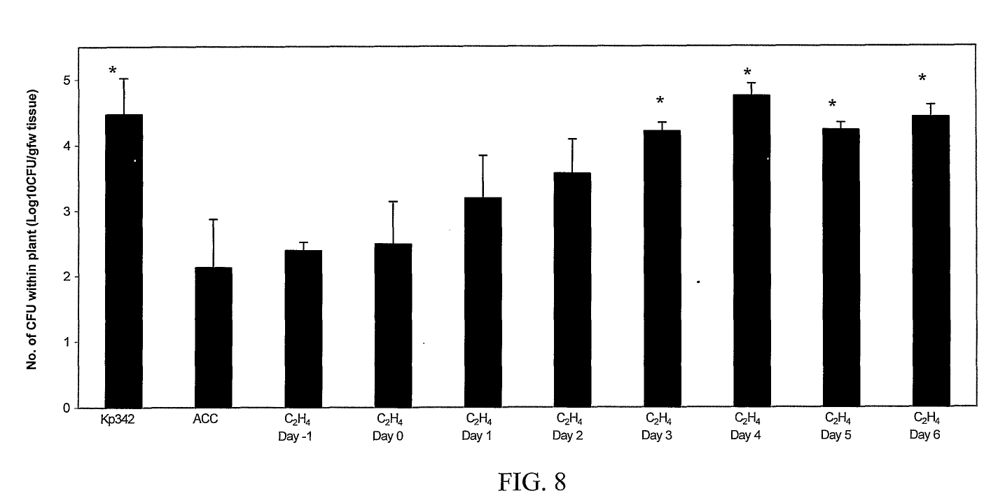

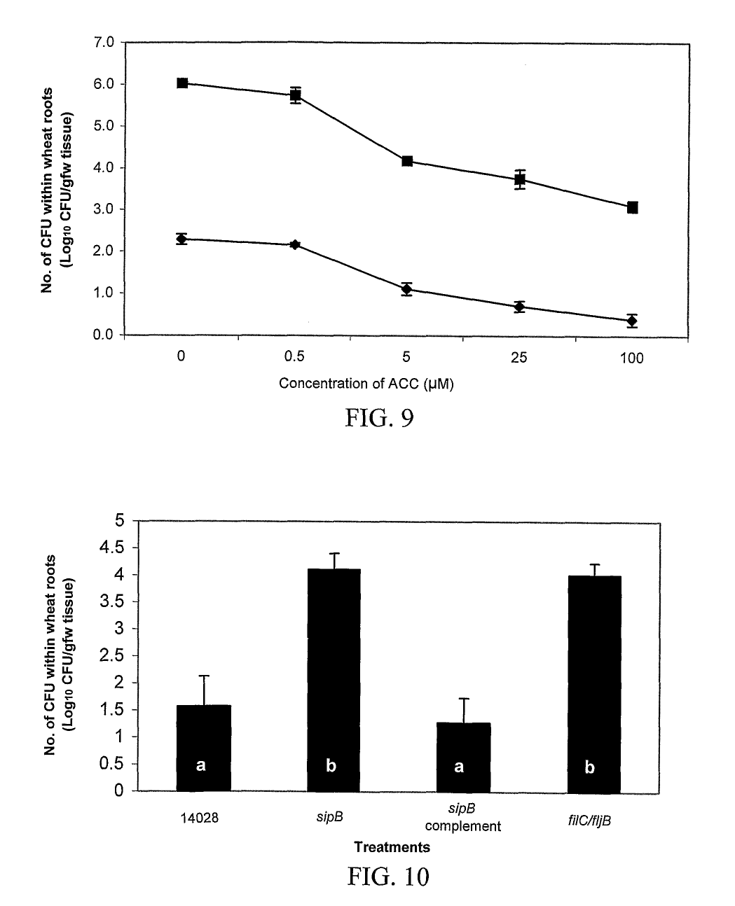

Ethylene has been extensively studied as a secondary messenger in the induction of a salicylic acid (SA)-independent plant defense pathway referred to as induced systemic resistance or ISR (Dong et al., 2003a; Knoester et al., 1998; Pieterse et al., 1998; Ton et al., 2001; Ton et al., 2002). Kp342 hypercolonized an ethylene-insensitive (sickle) M. truncatula mutant (FIG. 5). This mutant is also hypernodulated following inoculation with the nitrogen-fixing symbiont Sinorhizobium meliloti (Penmetsa and Cook 1997). Consistent with this result, addition of the ethylene precursor, 1-aminocyclopropane-1-carboxylic acid (ACC), to the growth media significantly reduced endophytic colonization in wild-type M. sativa by Kp342 and Salmonella enterica serovar Typhimurium strain 14028 (S. typhimurium) by three and four orders of magnitude, respectively (FIG. 6). The number of Kp342 cells within Medicago truncatula roots does not change significantly with ACC treatment until four days after inoculation. This evidence suggests that ACC does not inhibit invasion of Kp342 cells into the plant but triggers a response that can significantly lower the number of Kp342 cells four days after ACC treatment (FIG. 7). To test the effects of ethylene on Medicago truncatula before, during, and after inoculation a time-course experiment was conducted (FIG. 8). In this experiment, gaseous ethylene (C₂H₄) was used rather than ACC since addition of C₂H₄ was required each day for up to six days during the time course. No difference in endophytic colonization was observed in plants exposed to C₂H₄ or ACC for the same time period (FIG. 8). This time course experiment showed that ethylene must be applied to the plants prior to, or at the time of inoculation, for maximal inhibition of endophytic colonization (FIG. 8). These results further corroborate those of FIG. 7. That is, the effects of ethylene on endophytic colonization become significant 96 hours after ethylene exposure. To determine whether ethylene affects endophytic colonization in monocots, wheat seedlings were exposed to varying amounts of ACC and inoculated with Kp342 and 14028 (FIG. 9). ACC caused a decline in the number of Kp342 and 14028 cells within wheat roots of 1.85 and 1.2 orders of magnitude, respectively (FIG. 9).

To confirm that the effects observed with ACC were specific to ethylene production, a specific inhibitor of ethylene-mediated signaling, 1-methylcyclopropene (1-MCP) (Porat et al., 1999; Serek et al., 1995), reversed the reduction in endophytic colonization of alfalfa observed with ACC (FIG. 6). Also, treatment of plants with 1-MCP resulted in significantly higher endophytic colonization regardless of the presence or absence of exogenous ACC in M. truncatula (FIG. 6). These results suggest that endogenously produced ethylene limits the extent of endophytic colonization in M. truncatula but not in M. sativa.

### EXAMPLE 6

### Presence of Bacterial Extracellular Components Decreases Endophytic Colonization

Bacterial extracellular components, such as flagella, are known to induce plant defenses (Felix et al., 1999; Gomez-Gomez and Boller 2000). A Salmonella 14028 mutant lacking both flagellin genes, fliC and fliB, fails to produce flagella in culture. This mutant showed significantly higher endophytic colonization, consistent with the notion that Salmonella flagellar components are specifically recognized and induce plant defenses. Another extracellular component of enteric bacteria, the type III secretion system encoded by Salmonella pathogenicity island 1 (TTSS-SPI1), also affects endophytic colonization. The TTSS-SPI1 is a virulence factor that promotes invasion of mammalian cells and elicits fluid secretion and inflammation in animal models (Zhang et al., 2003). The sipB and spaS genes are encoded within SPI1. The spaS gene encodes a structural component of the type III secretion apparatus, while the sipB gene encodes a protein with dual functions. SipB is required for translocation of other effectors and has effector properties of its own (Collazo and Galan 1997). Furthermore, secretion of SipB is independent of bacterial-host cell contact and therefore is not necessarily concomitant with translocation to host cells (Collazo and Galan 1997). Mutations in spaS and sipB resulted in much higher levels of colonization in alfalfa roots (FIG. 6). When these mutants were complemented with a wild-type copy of the gene, the reduced colonization phenotype was restored (FIG. 6). Similar results were obtained with the sipB mutant on wheat seedlings (FIG. 10).

With the removal of these extracellular components, ethylene-mediated inhibition of endophytic colonization, although still significant, was greatly reduced compared to the wild-type strain (FIG. 6). ACC decreases endophytic colonization by over two orders of magnitude for the wild-type strain (FIG. 6) whereas, the ACC-induced decrease is only 0.5 to 1.1 orders of magnitude when the seedlings were inoculated with the spaS or double flagellin mutants, respectively (FIG. 6). The Salmonella sipB and double flagellin mutations also caused an increase of 2.5- and 2.4 orders of magnitude, respectively, in the number of Salmonella cells within wheat roots compared to wild-type Salmonella 14028. Complementation of the sipB mutant completely reversed the increase observed from the sipB mutation (FIG. 6).

### EXAMPLE 7

### Increased Endophytic Colonization in Host Genotypes with Diminished Plant Defense Responses

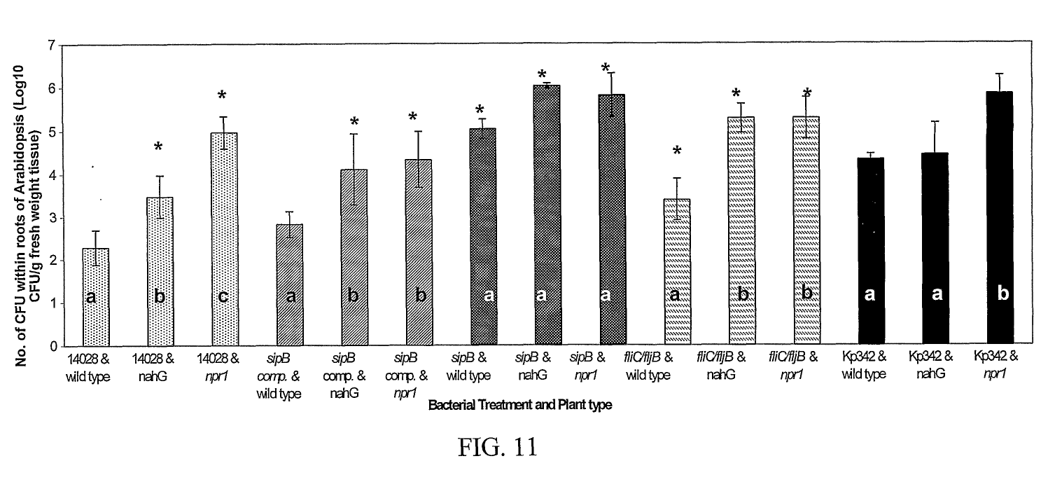

The importance of plant defenses on endophytic colonization were examined using Arabidopsis lines impaired in plant defense. Strain 14028, the sipB and double flagellin mutants of 14028, and Kp342 were individually inoculated onto the roots of Arabidopsis wild-type Col-0, a nahG transgenic plant, and an npr1 mutant (FIG. 11). The nahG transgenic plant produces a bacterial salicylate hydroxylase (Friedrich et al, 1995) that prevents the accumulation of salicylic acid in plants. The NPR1 protein regulates the DNA binding ability of transcription factors involved in plant defense (Despres et al., 2003; Mou et al., 2003), and the Arabidopsis npr1 mutant is disrupted in both SA-mediated and SA-independent defense responses (Ton et al., 2002).

Colonization by Kp342 was not significantly different on wild-type Arabidopsis compared with the nahG transgenic plants, suggesting that accumulation of SA is not important for restricting colonization by Kp342. However, colonization of the npr1 mutant by Kp342 was 1.5 orders of magnitude greater than in wild-type Arabidopsis. These data suggest that SA-independent defense responses (defective in the npr1 mutant) may contribute to reduced colonization by Kp342.

The interior colonization of Arabidopsis roots by 14028 was 1.2 to 2.7 orders of magnitude greater in the nahG transgenic and npr1 mutant, respectively (FIG. 11) compared to wild-type plants, suggesting both SA-dependent and SA-independent pathways are involved in restricting colonization. The roles of flagellin and TTSS-SPI1 in colonization were examined by mutational analysis. Both the Salmonella double flagellin mutant (fliClfljB) and the TTSS-SPI1 (sipB) mutants colonized the roots of wild-type Arabidopsis in significantly greater numbers than the wild-type strain 14028 (FIG. 11), supporting roles for both of these extracellular components in plant recognition.

Colonization by the flagella mutant was 1.9 orders of magnitude greater in the nahG transgenic and npr1 mutant than in wild-type plants (FIG. 11). For the nahG transgenic plants, these results are consistent with colonization behavior observed for 14028. However, no difference was observed in endophytic colonization of the npr1 mutant by 14028 or the flagella mutant but the wild type host was colonized significantly more by the flagella mutant compared to 14028. Equal colonization of the nahG transgenic and the npr1 mutant by the Salmonella flagella mutant imply that endophyte recognition and the subsequent defenses induced by flagella and largely SA-independent. That is, a plant defective in SA accumulation still allows more colonization by a flagella-defective endophyte, while a mutant defective in both SA-dependent and SA-independent responses fails to exhibit super-enhanced colonization (as was observed for wild-type bacteria).

In contrast, data obtained with the TTSS-SPI1 defective sipB mutant suggest that the lack of TTSS-SPI1 effectors permits the avoidance of SA-dependent and SA-independent responses. Whereas, colonization of wild-type plants was enhanced by the sipB TTSS-SPI1 mutation, colonization by sipB was not significantly different in nahG transgenic and npr1 mutants. Therefore, while colonization by a bacterium defective in TTSS-SPI1 was significantly enhanced in wild-type plants, it was unaffected by compromising both SA-dependent and SA-independent defense pathways in the host plant. These data suggest that a sipB-regulated TTSS-SPI1 effector(s) act downstream of SA and npr1 in this system. As predicted, the increased colonization observed with the sipB mutant was reversed when the mutant was complemented with the wild-type gene.

These data also support the notion that the TTSS-SPI1 of 14028 induces both the SA-mediated and SA-independent responses, in agreement with 14028 induction of the SA-mediated PR1 promoter.

### EXAMPLE 8

### Activation of a Promoter that Controls a Salicylic Acid-dependent Pathogenesis-related Gene Upon Endophyte Inoculation

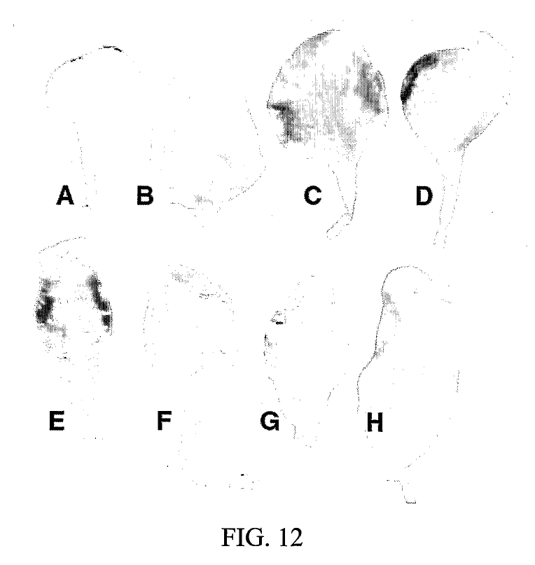

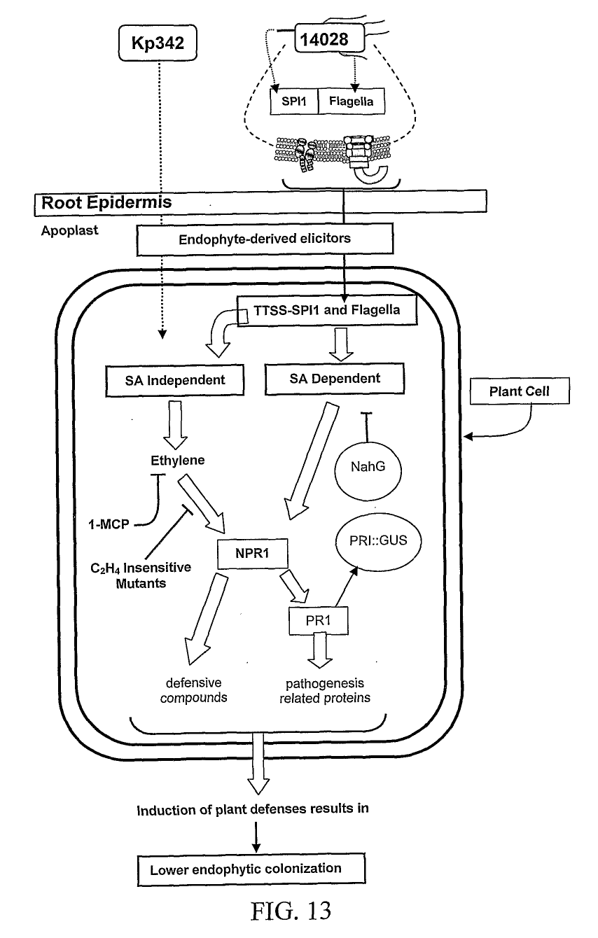

In support of the elicitation of plant defenses during endophytic colonization, the expression of the extensively studied plant defense response gene PR1 (Beilmann et al., 1992) was tested by inoculation of Arabidopsis thaliana PR1::GUS with our enteric endophtyes. The positive controls, application of salicylic acid or inoculation with the plant pathogen Pseudomonas syringae DC3000 PV288, strongly induced a PR1::GUS fusion in planta. Both positive controls rendered expected results in the GUS histochemical staining and GUS fluorogenic assays. Inoculation of roots with St14028 also induced PR1::GUS expression in distal leaves, displaying a GUS activity of 42 pmol 4-MU/mg protein/min, (FIG. 12). In contrast, inoculation of roots with the sipB mutant showed no GUS induction. Complementation of the sipB mutation restored GUS expression and activity (19 pmol 4-MU/mg protein/min) (FIG. 12). Negative controls, where plants where sprayed with H₂O, leaf infiltration with PBS, or root inoculation with PBS failed to induce PR1::GUS expression. Because the PR1 promoter is induced by the salicylic acid signaling pathway (Stone et al., 2000), these data suggest that the TTSS-SPI1 induce SA-mediated defense signaling. Unlike 14028, inoculation with Kp342 did not result in PR1::GUS expression suggesting that this endophyte does not induce SA-dependent defense responses (data not shown), consistent with Klebsiella lacking flagella and TTSS-SPI1.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

### WO 00/065037

Ahmer, B. M. M., van Reeuwijk, J., Watson, P. R., Wallis, T. S., Heffron, F. (1999) “Salmonella sirA is a global regulator of genes mediating enteropathogenesis” Mol. Microbiol. 31: 971-982.
Beilmann, A., Albrecht, K., Schultze, S., Wanner, G., Pfitzner, U. M. (1992) “Activation of a truncated PR-I promoter by endogenous enhancers in transgenic plants” Plant Mol. Biol. 18: 65-78.
Beuchat, L. R., Ward, T. E., and Pettigrew, C. A. (2001) “Comparison of chlorine and a prototype produce wash product for effectiveness in killing salmonella and escherichia coli o157: H7 on alfalfa seeds” Journal of Food Protection 64: 152-158.
Boddey, R. M. and Chalk, P. M. (1983) “The ¹⁵N-isotope dilution technique applied to the estimation of biological nitrogen fixation associated with Paspalum notatum cv. Batatais in the field” Soil. Biol. Biochem. 15: 25-32.
Boddey, R. M., Baldani, V. L. D., Baldani, J. I., and Döbereiner, J. (1986a) “Effect of inoculation of Azospirillum spp. on nitrogen accumulation by field-grown wheat” Plant Soil 95: 109-121.
Boddey, R. M. and Victoria, R. L. (1986b) “Estimation of biological nitrogen fixation associated with Brachiaria and Paspalum grasses using ¹⁵N-labelled organic matter and fertilizer” Plant Soil 90: 265-292.
Boddey, R. M., Urquiaga, S., Alves, B. J. R., and Reis, V. (2003) “Endophytic nitrogen fixation in sugarcane: Present knowledge and future applications” Plant and Soil 252: 139-149.
Bremer, E., Janzen, H. H., and Gilbertson, C. (1995) “Evidence against associative N₂ fixation as a significant n source in long-term wheat plots” Plant Soil 175: 13-19.
Cannon, F. C., Riedel, G. E., and Ausubel, F. M. (1979) “Overlapping sequences of K. pneumoniae nif DNA cloned and characterized” Mol. Gen. Genet. 174: 59-66.
Cao, H., Li, X., Dong, X. N. (1998) “Generation of broad-spectrum disease resistance by overexpression of an essential regulatory gene in systemic acquired resistance” Proc. Natl. Acad. Sci. USA 95: 6531-6536.
Chelius, M. K. and Triplett, E. W. (2000) “Immunolocalization of dinitrogenase reductase produced by Klebsiella pneumoniae in association with Zea mays L.” Appl. Environ. Microbiol. 66: 783-787.
Collazo, C. M., and Galan, J. E. (1997) “The invasion-associated type iii system of salmonella typhimurium directs the translocation of sip proteins into the host cell” Molecular Microbiology 24: 747-756.
Despres, C., Chubak, C., Rochon, A., Clark, R., Bethune, T., Desveaux, D., and Fobert, P. R., (2003) “The arabidopsis npr1 disease resistance protein is a novel cofactor that confers redox regulation of DNA binding activity to the basic domain/leucine zipper transcription factor tga1” Plant Cell 15: 2181-2191.
Dixon, R., Cheng, Q., Shen, G. -F., Day, A., and Dowson-Day, M. (1997) “Nif gene transfer and expression in chloroplasts: Prospects and problems” Plant Soil 194: 193-203.
Dong, Y., Chelius, M. K., Brisse, S., Kozyrovska, N., Kovtunovych, G., Podschun, R., and Triplett, E. W. (2003a) “Comparisons between two Klebsiella: the plant endophyte K pneumoniae 342 and a clinical isolate, K. pneumoniae MGH78578” Symbiosis 35: 247-259.
Dong, Y., Iniguez, A. L., Ahmer, B. M. M., and Triplett, E. W. (2003b) “Kinetics and strain specificity of rhizosphere and endophytic colonization by enteric bacteria on seedlings of Medicago sativa and Medicago trucatula” Appl. Environ. Microbiol. 69: 1783-1790.
Dong, Y., Iniguez, A. L., and Triplett, E. W. (2003c) “Quantitative assessments of the host range and strain specificity of endophytic colonization by Klebsiella pneumoniae 342” Plant Soil 257: 49-59.
Felix, G., Duran, J. D., Volko, S., and Boller, T. (1999) “Plants have a sensitive perception system for the most conserved domain of bacterial flagellin” Plant Journal 18: 265-276.
Friedrich, L., Vernooij, B., Gaffney, T., Morse, A., and Ryals, J. (1995) “Characterization of tobacco plants expressing a bacterial salicylate hydroxylase gene” Plant Molecular Biology 29: 959-968.
Gomez-Gomez, L., and Boller, T. (2000) “Fls2: An Irr receptor-like kinase involved in the perception of the bacterial elicitor flagellin in Arabidopsis” Molecular Cell 5: 1003-1011.
Hall, A. E., Findell, J. L., Schaller, G. E., Sisler, E. C., Bleecker, A. B. (2000) “Ethylene perception by the ERSI protein in Arabidopsis” Plant Physiol. 123: 1449-1457.
Hurek, T., Handley, L. L., Reinhold-Hurek, B., and Piche, Y. (2002) “Azoarcus grass endophytes contribute fixed nitrogen to the plant in an unculturable state” Mol. Plant-Microbe Interact. 15: 233-242.
Jefferson, R. A., Kavanagh, T. A., and Bevan, M. W. (1987) “Gus fusions—beta-glucuronidase as a sensitive and versatile gene fusion marker in higher-plants” Embo. Journal 6: 3901-3907.
Kahn, M. L., Parra-Colmenares, A., Ford, C. L., Kaser, F., McCaskill, D., and Ketchum, R. E. (2002) “A mass spectrometry method for measuring ¹⁵N incorporation into pheophytin” Anal. Biochem. 307: 219-225.
Kapulnik, Y., Feldman, M., Okon, Y., and Henis, Y. (1985) “Contribution of nitrogen fixed by Azospirillum to the N nutrition of spring wheat in Israel” Soil Biol Biochem. 17: 509-515.
Knoester, M., van Loon, L. C., van den Heuvel, J., Hennig, J., Bol, J. F., and Linthorst, H. J. M. (1998) “Ethylene-insensitive tobacco lacks nonhost resistance against soil-borne fungi” Proc. Natl. Acad. Sci. USA 95: 1933-1937.
Kucey, R. M. N. (1988) “Alteration of size of wheat root systems and nitrogen fixation by associative nitrogen-fixing bacteria under field conditions” Can. J. Microbiol 34: 735-739.
Kunkel, B. N., Bent, A. F., Dahlbeck, D., Innes, R. W., and Staskawicz, B. J., (1993) “Rps2, an Arabidopsis disease resistance locus specifying recognition of pseudomonas-syringae strains expressing the avirulence gene avrrpt2” Plant Cell 5: 865-875.
Lethbridge, G., and Davidson, M. S. (1983) “Root-associated nitrogen-fixing bacteria and their role in the nitrogen nutrition of wheat estimated by ¹⁵N isotope-dilution” Soil Biol. Biochem. 15: 365-374.
Miranda, C. H. B. and Boddey, R. M. (1987) “Estimation of biological nitrogen fixation associated with 11 ecotypes of Panicum maximum grown in nitrogen-15-labeled soil” Agron. J. 79: 558-563.
Mou, Z., Fan, W. H., and Dong, X. N. (2003) “Inducers of plant systemic acquired resistance regulate npr1 function through redox changes” Cell 113: 935-944.
Penmetsa, R. V., and Cook, D. R. (1997) “A legume ethylene-insensitive mutant hyperinfected by its rhizobial symbiont” Science 275: 527-530.
Pieterse, C. M. J., van Wees, S. C. M., van Pelt, J. A., Knoester, M., Laan, R., Gerrits, N., Weisbeek, P. J., and van Loon, L. C. (1998) “A novel signaling pathway controlling induced systemic resistance in Arabidopsis” Plant Cell 10: 1571-1580.
Porat, R., Weiss, B., L. Cohen, L., Daus, A., Goren, R. and Droby, S. (1999) “Effects of ethylene and 1-methylcyclopropene on the postharvest qualities of ‘shamouti’ oranges” Postharvest Biology and Technology 15: 155-163.
Proctor, M. E., Hamacher, M., Tortorello, M. L., Archer, J. R., and Davis, J. P. (2001) “Multistate outbreak of salmonella serovar muenchen infections associated with alfalfa sprouts grown from seeds pretreated with calcium hypochlorite” J. Clin. Microbiol. 39: 3461-3465.
Reece, K. S. and Phillips, G. J. (1995) “New plasmids carrying antibiotic-resistance cassettes” Gene 165: 141-142.
Rennie, R. J., deFreitas, J. R., Ruschel, A. P., and Vose, P. V. (1983) “¹⁵N isotope dilution to quantify dinitrogen (N₂) fixation associated with Canadian and Brazilian wheat” Can. J. Bot. 61: 1667-1671.
Reuber, T. L., Plotnikova, J. M., Dewdney, J., Rogers, E. E., Wood, W. Ausubel, F. M. (1998) “Correlation of defense gene induction defects with powdery mildew susceptibility in Arabidopsis enhanced disease susceptibility mutants” Plant J. 16(4): 473-485.
Riggs, P. J., Chelius, M. K., Iniguez, A. L., Kaeppler, S. M., and Triplett, E. W. (2001) “Enhanced maize productivity by inoculation with diazotrophic bacteria” Austr. J. Plant Physiol. 28: 829-836.
Schmitt, C. K., Ikeda, J. S., Darnell, S. C., Watson, P. R., Bispham, J., Wallis, T. S., Weinstein, D. L., Metcalf, E. S., and O'Brien, A. D. (2001) “Absence of all components of the flagellar export and synthesis machinery differentially alters virulence of salmonella enterica serovar typhimurium in models of typhoid fever, survival in macrophages, tissue culture invasiveness, and calf enterocolitis” Infection and Immunity 69: 5619-5625.
Scupham, A. J., and Triplett, E. W. (1997) “Isolation and characterization of the UDP-glucose 4′-epimerase gene, galE, from Brucella abortus S2308” Gene 202: 53-59.
Serek, M., Tamari, G., Sisler, E. C., and Borochov, A. (1995) “Inhibition of ethylene-induced cellular senescence symptoms by 1-methylcyclopropene, a new inhibitor of ethylene action” Physiologia Plantarum 94: 229-232.
Sevilla, M., Burris, R. H., Gunapala, N., and Kennedy, C. (2001) “Comparison of benefit to sugarcane plant growth and ¹⁵N₂ incorporation following inoculation of sterile plants with Acetobacter diazotrophicus wild-type and Nif mutant strains” Mol. Plant-Microbe Interact. 14: 358-366.
Shrestha, R. K. and Ladha, J. K. (1996) “Genotypic variation in promotion of rice dinitrogen fixation as determined by nitrogen-15 dilution” Soil Sci. Soc. Am. J. 60: 1815-1821.
Stone, J. M., Heard, J. E., Asai, T., and Ausubel, F. M. (2000) “Simulation of fungal-mediated cell death by fumonisin b1 and selection of fumonisin b1-resistant (fbr) arabidopsis mutants” Plant Cell 12: 1811-1822.
Suárez, M., Ferrer, E., Garrido-Pertierra, A., and Martin, M. (1995) “Purification and characterization of the 3-hydroxybenzoate-6-hydroxylase from Klebsiella pneumoniae” FEMS Microbiol. Lett. 126: 283-290.
Sundaresan, V., Springer, P., Volpe, T., Haward, S., Jones, J. D. G., Dean, C., Ma, H., and Martienssen, R. (1995) “Patterns of gene-action in plant development revealed by enhancer trap and gene trap transposable elements” Genes & Development 9: 1797-1810.
Taormina, P. J., Beuchat, L. R., and Slutsker, L. (1999) “Infections associated with eating seed sprouts: An international concern” Emerging Infectious Diseases 5: 626-634.
Ton, J., Davison, S., Van Wees, S. C. M., Van Loon, L. C., and Pieterse, C. M. J. (2001) “The arabidopsis isr1 locus controlling rhizobacteria-mediated induced systemic resistance is involved in ethylene signaling” Plant Physiology 125: 652-661.
Ton, J., De Vos, M., Robben, C., Buchala, A., Metraux, J. P., Van Loon, L. C., and Pieterse, C. M. J. (2002) “Characterization of arabidopsis enhanced disease susceptibility mutants that are affected in systemically induced resistance” Plant Journal 29: 11-21.
Urquiaga, S., Cruz, K. H. S., and Boddey, R. M. (1992) “Contribution of nitrogen fixation to sugar cane—Nitrogen-15 and nitrogen-balance estimates” Soil Sci. Soc. Am. J. 56: 105-114.
Wang, R. F., and Kushner, S. R. (1991) “Construction of versatile low-copy-number vectors for cloning, sequencing and gene-expression in escherichia-coli” Gene 100: 195-199.
Weissinger, W. R., and Beuchat, L. R. (2000) “Comparison of aqueous chemical treatments to eliminate salmonella on alfalfa seeds” J. Food Protection 63: 1475-1482.
Weissinger, W. R., McWatters, K. H. and Beuchat, L. R. (2001) “Evaluation of volatile chemical treatments for lethality to salmonella on alfalfa seeds and sprouts” J. of Food Protection 64: 442-450.
Wu, P., Zhang, G., Ladha, J. K., McCouch, S. R., and Huang, N. (1995) “Molecular-marker-facilitated investigation on the ability to stimulate N₂ fixation in the rhizosphere by irrigated rice plants” Theor. Appl. Genetic. 91: 1177-1183.
Zhang, S. P., Kingsley, R. A., Santos, R. L., Andrews-Polymenis, H., Raffatellu, M., Figueiredo, J., Nunes, J., Tsolis, R. M., Adams, L. G., and Baumler, A. J. (2003) “Molecular pathogenesis of salmonella enterica serotype typhimurium-induced diarrhea” Infection and Immunity 71: 1-12.

1-108. (canceled)

109. A method for enhancing nitrogen fixation in a plant, said method comprising:
a) inoculating a plant seed with an effective amount of a nitrogen fixing endophytic bacteria, and growing a plant from said plant seed; or
b) inoculating said plant with an effective amount of a nitrogen fixing endophytic bacteria to colonize said plant; or
c) obtaining a plant seed from a plant colonized with a seed borne nitrogen fixing endophytic bacteria, and growing a plant from said plant seed.

110. The method according to claim 109, wherein said nitrogen fixing bacteria is originally isolated from a nitrogen efficient plant.

111. The method according to claim 109, wherein said nitrogen fixing bacteria does not express one or more bacterial extracellular components or expresses lower levels of said one or more extracellular components.

112. The method according to claim 111, wherein said nitrogen fixing bacteria lack one or more sip, spa, or fli genes or express one or more mutant sip, spa, or fli genes encoding a non-functional gene product, for example, wherein said sip gene is sipB, or said spa gene is spaS, or said fli gene is fliC or fliB.

113. The method according to claim 109, wherein said plant is a non-leguminous plant or an agronomically important grass, such as wheat, rice, maize, barley, oat, sorghum, or rye.

114. The method according to claim 109, wherein said plant seed is inoculated with said nitrogen fixing bacteria by submerging said plant seed in a suspension of said nitrogen fixing bacteria.

115. The method according to claim 109, wherein said bacteria is Klebsiella pneumoniae, or a strain thereof, such as Klebsiella pneumonia strain Kp342.

116. The method according to claim 109, wherein said plant is resistant to colonization or infection by a bacterial pathogen.

117. The method according to claim 116, wherein said plant expresses one or more defense responses against said bacterial pathogen.

118. The method according to claim 117, wherein said defense response can be induced in said plant.

119. The method according to claim 117, wherein said defense response is an ethylene-mediated defense response or said defense response is a salicyclic acid-mediated or a salicyclic acid-independent defense response.

120. The method according to claim 116, wherein said plant expresses or overexpressses an NPR1 gene, such as an NPR1 gene that encodes a polypeptide having the sequence shown in SEQ ID NO: 4, 6, or 8, or a biologically active fragment of any of said sequences.

121. The method according to claim 116, wherein said plant is resistant to colonization or infection by Salmonella.

122. The method according to claim 109, wherein the roots of said plant are inoculated with said nitrogen fixing bacteria.

123. A composition of matter comprising:
a) an isolated nitrogen fixing endophytic bacteria, wherein said nitrogen fixing bacteria is originally isolated from a nitrogen efficient plant and is seed borne; or
b) a plant that is resistant to colonization by a bacterial pathogen, wherein said plant is engineered to express a defense response against said pathogen.

124. The nitrogen fixing bacteria according to claim 123, wherein said nitrogen fixing bacteria does not express one or more bacterial extracellular components or expresses lower levels of said one or more extracellular components.

125. The nitrogen fixing bacteria according to claim 124, wherein said nitrogen fixing bacteria lack one or more sip, spa, or fli genes or express one or more mutant sip, spa, or fli genes encoding a non-functional gene product, for example, wherein said sip gene is sipB, or said spa gene is spaS, or said fli gene is fliC or fliB.

126. The plant according to claim 123, wherein said defense response can be induced in said plant upon exposure of said plant to a selected substance or condition.

127. The plant according to claim 123, wherein said plant overexpresses an NPR1 gene, such as NPR1 gene that encodes a polypeptide having the sequence shown in SEQ ID NO: 4, 6, or 8, or a biologically active fragment of any of said sequences.

128. The plant according to claim 123, wherein said plant is a monocotyledonous plant or a dicotyledonous plant.

129. A method for:
a) increasing the number of free-living nitrogen-fixing endophytic bacteria in a plant, said method comprising preparing mutant nitrogen-fixing endophytic bacteria that are resistant to plant defense responses and inoculating said plant with said mutant bacteria; or
b) eliminating or decreasing the number of bacterial pathogens residing on or within plant tissue, said method comprising inducing a plant defense response to one or more bacterial pathogens residing within said plant tissue.

130. The method according to claim 129, wherein said mutant bacteria are prepared by exposing nitrogen-fixing endophytic bacteria to extracts of tissue from a plant whose defense responses have been induced.

131. The method according to claim 130, wherein said induced plant defense responses are ethylene-mediated defense responses or said defense response is a salicylic acid-mediated or salicylic acid-independent defense response.

132. The method according to claim 130, further comprising selecting bacteria that survive exposure to said extracts of tissue from plants.

133. The method according to claim 130, wherein said bacteria exposed to said tissue extracts are bacteria that do not express one or more bacterial extracellular components or expresses lower levels of said one or more extracellular components.

134. The method according to claim 133, wherein said nitrogen fixing bacteria lack one or more sip, spa, or fli genes or express one or more mutant sip, spa, or fli genes encoding a non-functional gene product, for example, wherein said sip gene is sipB, or said spa gene is spaS, or said fli gene is fliC or fliB.

135. The method according to claim 130, wherein said mutant bacteria are resistant to salicyclic acid-mediated or salicyclic acid-independent plant defense responses.

136. The method according to claim 130, wherein said plant is a non-leguminous plant or an agronomically important grass, such as wheat, rice, maize, barley, oat, sorghum, or rye.

137. The method according to claim 131, wherein said plant defense response is induced by treating or exposing said plant to a chemical that induces said defense response.


SEQUENCE LISTING (10 sequences)

SEQ ID NO: 1 - DNA, 29 bp, K. oxytoca
gcctgcagatgaccatgcgtcaatgcgcc

SEQ ID NO: 2 - DNA, 29 bp, K. oxytoca
gcgaattccgcgttttcttcggcggcggt

SEQ ID NO: 3 - DNA, 2090 bp, Brassica napus
tggatctcaagtctttcgattgataagtattcttcgttgattcggtccttctttcttggagtcttcacttctcaccatcggatctctgtgacctttgatggagaccattgctggatttgatgatttctatgagatcagcagcactagcttcctcgccgcaccggcgccaaccgataactccggatcatccaccgtctacccgacggagcttttcaccagacccgaggtatccgcgtttcaactcctctccaacagcctcgagtccgtcttcgactcgccggaagcgttctacagcgacgccaagcttgttctctccgacgacaaggaagtatccttccaccgttgcattctctcggcgagaagcctcttcttcaaggccgctttgacagccgccgagaaggtgcagaagtccacccccgtgaagctcgagctgaagacactcgcggcggaatacgacgtcgggttcgattctgtggtggctgttctggcgtacgtttacagcggcagagtgaggccgcctccgaagggagtttctgaatgcgcagacgagagctgctgccacgtggcgtgccgtccggctgtggatttcatggtggaggttctctacttggctttcgtcttccagattcaggaactggttaccatgtatcagaggcatttactggatgttgtagacaaagttatcatagaagacactttggtcgtcctcaagcttgctaacatctgcggtaaagcgtgcaagaagctattcgataagtgcagagagatcattgtcaagtctaacgtggatgttgttactctaaagaagtcattgcctgaggacattgccaagcaagtaatcgatatccgcaaagagctcggcttggaggtagctgaaccagagaaacatgtctccaacatacacaaggcgcttgagtcagacgatcttgaccttgtcgttatgcttttgaaagagggccacacgaatctagacgaagcgtatgctctccattttgctgttgcgtattgcgatgagaagacagcgaggaatctcctggaactggggtttgcggatgtcaaccggagaaacccgagagggtacacggtaattcacgtcgctgcgatgaggaaagagccgacactgatagcattgttgttgacgaaaggggctaatgcattagaaatgtctttggacgggagaactgctctgttgatcgcgaaacaagtcactaaggcggccgagtgttgtattctggagaaagggaagttagctgccaaaggcggagtatgtgtagagatactcaagcaaccagacaacacacgagaaccatttcctgaagatgtttctccctcccttgcagtggctgctgatcaattcaagataaggttgattgatcttgaaaacagagttcaaatggctcgatgtctctatccaatggaagcacaagttgcaatggatttcgcccgaatgaagggaacacgcgagtttgtcgtgacgacagcaactgacctacacatggaacctttcaagttcgtagaaatgcatcagagtagactaacagcgctttctaaaactgtggaattcgggaaacgcttcttcccacgctgttcgaaagtgctcgatgatattgtggactctgaggacttgactatactggctctcgtagaagaagacactcctgagcaacgacaacaaaagaggcagaggttcatggaaatacaggagattgttcaaatggcgtttagtaaagacaaggaggatcttggaaagtcgtctctctcagcttcgtcttcttccacatccaaattaactggtaaaaagaggtctattgctaaaccctctcaccggcgtcggtgacactatttgttcggggtaatatttgatgtaccatatatatatatttgagttatcatgtgtcttagtgtctctgtctattgttggctcagtcatatagttttgttcgtcattttgcatcctgtgtattccgcagctttcggtgtgtttgaagcaaatgctgtaaccttttgaatctgtgatatatctattataataaaccatgatgggtgttacagaaaaaaaaaaaaaaaaaaaaaaaaaaaaaaaaaaaaaa

SEQ ID NO: 4 - DNA, 1740 bp, Brassica napus
  exon (1)..(1740)
atggagaccattgctggatttgatgatttctatgagatcagcagcactagcttcctcgccgcaccggcgccaaccgataactccggatcatccaccgtctacccgacggagcttttcaccagacccgaggtatccgcgtttcaactcctctccaacagcctcgagtccgtcttcgactcgccggaagcgttctacagcgacgccaagcttgttctctccgacgacaaggaagtatccttccaccgttgcattctctcggcgagaagcctcttcttcaaggccgctttgacagccgccgagaaggtgcagaagtccacccccgtgaagctcgagctgaagacactcgcggcggaatacgacgtcgggttcgattctgtggtggctgttctggcgtacgtttacagcggcagagtgaggccgcctccgaagggagtttctgaatgcgcagacgagagctgctgccacgtggcgtgccgtccggctgtggatttcatggtggaggttctctacttggctttcgtcttccagattcaggaactggttaccatgtatcagaggcatttactggatgttgtagacaaagttatcatagaagacactttggtcgtcctcaagcttgctaacatctgcggtaaagcgtgcaagaagctattcgataagtgcagagagatcattgtcaagtctaacgtggatgttgttactctaaagaagtcattgcctgaggacattgccaagcaagtaatcgatatccgcaaagagctcggcttggaggtagctgaaccagagaaacatgtctccaacatacacaaggcgcttgagtcagacgatcttgaccttgtcgttatgcttttgaaagagggccacacgaatctagacgaagcgtatgctctccattttgctgttgcgtattgcgatgagaagacagcgaggaatctcctggaactggggtttgcggatgtcaaccggagaaacccgagagggtacacggtaattcacgtcgctgcgatgaggaaagagccgacactgatagcattgttgttgacgaaaggggctaatgcattagaaatgtctttggacgggagaactgctctgttgatcgcgaaacaagtcactaaggcggccgagtgttgtattctggagaaagggaagttagctgccaaaggcggagtatgtgtagagatactcaagcaaccagacaacacacgagaaccatttcctgaagatgtttctccctcccttgcagtggctgctgatcaattcaagataaggttgattgatcttgaaaacagagttcaaatggctcgatgtctctatccaatggaagcacaagttgcaatggatttcgcccgaatgaagggaacacgcgagtttgtcgtgacgacagcaactgacctacacatggaacctttcaagttcgtagaaatgcatcagagtagactaacagcgctttctaaaactgtggaattcgggaaacgcttcttcccacgctgttcgaaagtgctcgatgatattgtggactctgaggacttgactatactggctctcgtagaagaagacactcctgagcaacgacaacaaaagaggcagaggttcatggaaatacaggagattgttcaaatggcgtttagtaaagacaaggaggatcttggaaagtcgtctctctcagcttcgtcttcttccacatccaaattaactggtaaaaagaggtctattgctaaaccctctcaccggcgtcggtga

SEQ ID NO: 5 - DNA, 1767 bp, Nicotiana tabacum
atggataatagtaggactgcgttttctgattcgaatgacatcagcggaagcagtagtatatgctgcatcggcggcggcatgactgaatttttctcgccggagacttcgccggcggagatcacttcactgaaacgcctatcggaaacactggaatctatcttcgatgcgtctttgccggagtttgactacttcgccgacgctaagcttgtggtttccggcccgtgtaaggaaattccggtgcaccggtgcattttgtcggcgaggagtccgttctttaagaatttgttctgcggtaaaaaggagaagaatagtagtaaggtggaattgaaggaggtgatgaaagagcatgaggtgagctatgatgctgtaatgagtgtattggcttatttgtatagtggtaaagttaggccttcacctaaagatgtgtgtgtttgtgtggacaatgactgctctcatgtggcttgtaggccagctgtggcattcctggttgaggttttgtacacatcatttacctttcagatctctgaattggttgacaagtttcagagacacctactggatattcttgacaaaactgcagcagacgatgtaatgatggttttatctgttgcaaacatttgtggtaaagcatgcgagagattgctttcaagctgcattgagattattgtcaagtctaatgttgatatcataacccttgataaagccttgcctcatgacattgtaaaacaaattactgattcacgagcggaacttggtctacaagggcctgaaagcaacggttttcctgataaacatgttaagaggatacatagggcattggattctgatgatgttgaattactacaaatgttgctaagagaggggcatactaccctagatgatgcatatgctctccattatgctgtagcgtattgcgatgcaaagactacagcagaacttctagatcttgcacttgctgatattaatcatcaaaattcaaggggatacacggtgctgcatgttgcagccatgaggaaagagcctaaaattgtagtgtcccttttaaccaaaggagctagaccttctgatctgacatccgatggaagaaaagcacttcaaatcgccaagaggctcactaggcttgtggatttcagtaagtctccggaggaaggaaaatctgcttcgaatgatcggttatgcattgagattctggagcaagcagaaagaagagaccctctgctaggagaagcttctgtatctcttgctatggcaggcgatgatttgcgtatgaagctgttataccttgaaaatagagttggcctggctaaactcctttttccaatggaagctaaagttgcaatggacattgctcaagttgatggcacttctgagttcccactggctagcatcggcaaaaagatggctaatgcacagaggacaacagtagatttgaacgaggctcctttcaagataaaagaggagcacttgaatcggcttagagcactctctagaactgtagaacttggaaaacgcttctttccacgttgttcagaagttctaaataagatcatggatgctgatgacttgtctgagatagcttacatggggaatgatacggcagaagagcgtcaactgaagaagcaaaggtacatggaacttcaagaaattctgactaaagcattcactgaggataaagaagaatatgataagactaacaacatctcctcatcttgttcctctacatctaagggagtagataagcccaataagctcccttttaggaaatag

SEQ ID NO: 6 - DNA, 1767 bp, Nicotiana tabacum; identical to SEQ ID NO: 5
  exon (1)..(1767)

SEQ ID NO: 7 - DNA, 2154 bp, Zea mays
gtcgtagtggtccgggtccggcacaagtaggggctcgcgtcttgcgcttggcagttgtgggaagccatggagccgtcgtcgtccatcacgttcgcgtcgtcgtcgtcgtacctgtccaacggctcgagcccctgttccgtcgcgctgccgccgccagggccgccccagactcccccgttgcctgccggccaggggtggggtggtggagtcgctgccgcagggagcggaggcagcgtggaggccgtgagcctgaaccggctcagcaaaaacctcgagcggctgctcctcgacccggacctagactgcagcgacgccgacgtcgatgtgcccgacggtgggccgcccgtacccatccaccgctgcatccttgccgcacgcagcgacttcttctacgacctcttcgccgctcgcggccgcgcaggggcagcgcgcggtgatgcggccgccggcgccggagtagccgcggagggggctgccagtggaaggccgcggtacaagatggaggatctcgttcccgccggccgcgtggggcgcgaggccttccaggcgtttctggggtacctgtacaccggcaagctccggccggcaccggtcgacgtggtgtcttgtgctgacccagtgtgccatcacgattcgtgcccgccggccatcaggtccgcggtcgagctcatgtacgcggcgtgtaccttcaagatccccgagctcacctcgctcttccagcgccggcttcttaattttgtagacaagactctagtggaggatgttattcctattctggaagttgcttcccactcagggctgactcaagtgatcgacaaatgtattcaaaggattgctagatcagatctcgacgatatatctttggataaggagctccctccagaagcagttgatgagataaaaaatttgcgcaagaagtcacaaactgctgatggtgatacgttcatttcggaccctgtgcatgagaaaagagtcagaagaatccacagggcacttgactctgatgatgttgagcttgtgaagttgcttcttaatgagtccgacatcacattagatgatgccaacgcattacactatgctgcttcttactgtgatcctaaagttgtctcagagctgttagatttggcaatggctaacttaaatttgaagaatagccgtgggtacacagcactccacttggctgctatgaggagagaaccagctataatcatgtgtctccttaacaaaggggcaaatgtgtcacaactgacagctgatggcaggagcgcaattggtatttgtcggaggttaacaagagcaaaagactacaatacaaagatggagcagggtcaagaatcaaataaagataggctgtgtatagatattctagagagggagatgatgcggaatcctatggcggtggaagatgccgtcacctcgcctttgttggcagatgatcttcacatgaagcttctctacctggaaaacagagttgcatttgctagattgttctttcctgctgaagccaaggtcgccatgcaaatcgcacaagcagacaccacagaagaattcggcggtatagttgcagttgcagcaagcacttctggtaaactgagggaggtggaccttaatgagacgccagtgacacaaaacaaaaggctccgttcaagggtagatgcactgatgaaaacagtggagctgggccgtcggtacttcccgaactgctcgcaggtgctggacaagttcctggaggacgatctgccggaaggtctggaccagttctacctccagaggggcacagccgatgagcagaaggtgaagaggatgcgcttctgcgagctgaaagaggacgtgctgaaggcgtttagcaaggacaaggcggagggcagcgtgttctcgggcctgtcctcgtcgtcgtcgtgctcgccgccccagaagtatgcccagaggtgatcaaggcaccagtttttgccgtatagtttgttatcatggtcttcgagacttggacccggacagcatatagggacatgtacacctgtgtatgtatagtgcttacaattggcgtaagtagaactatatgtatggaacataaggaaacatggcaggaacaccgtgcaaaaagatgaaaagatggccgaagtgctctatgcgaaaaaaaaaaaaaaaaaaaaaaaa

SEQ ID NO: 8 - DNA, 1866 bp, Zea mays
  exon (1)..(1866)
atggagccgtcgtcgtccatcacgttcgcgtcgtcgtcgtcgtacctgtccaacggctcgagcccctgttccgtcgcgctgccgccgccagggccgccccagactcccccgttgcctgccggccaggggtggggtggtggagtcgctgccgcagggagcggaggcagcgtggaggccgtgagcctgaaccggctcagcaaaaacctcgagcggctgctcctcgacccggacctagactgcagcgacgccgacgtcgatgtgcccgacggtgggccgcccgtacccatccaccgctgcatccttgccgcacgcagcgacttcttctacgacctcttcgccgctcgcggccgcgcaggggcagcgcgcggtgatgcggccgccggcgccggagtagccgcggagggggctgccagtggaaggccgcggtacaagatggaggatctcgttcccgccggccgcgtggggcgcgaggccttccaggcgtttctggggtacctgtacaccggcaagctccggccggcaccggtcgacgtggtgtcttgtgctgacccagtgtgccatcacgattcgtgcccgccggccatcaggtccgcggtcgagctcatgtacgcggcgtgtaccttcaagatccccgagctcacctcgctcttccagcgccggcttcttaattttgtagacaagactctagtggaggatgttattcctattctggaagttgcttcccactcagggctgactcaagtgatcgacaaatgtattcaaaggattgctagatcagatctcgacgatatatctttggataaggagctccctccagaagcagttgatgagataaaaaatttgcgcaagaagtcacaaactgctgatggtgatacgttcatttcggaccctgtgcatgagaaaagagtcagaagaatccacagggcacttgactctgatgatgttgagcttgtgaagttgcttcttaatgagtccgacatcacattagatgatgccaacgcattacactatgctgcttcttactgtgatcctaaagttgtctcagagctgttagatttggcaatggctaacttaaatttgaagaatagccgtgggtacacagcactccacttggctgctatgaggagagaaccagctataatcatgtgtctccttaacaaaggggcaaatgtgtcacaactgacagctgatggcaggagcgcaattggtatttgtcggaggttaacaagagcaaaagactacaatacaaagatggagcagggtcaagaatcaaataaagataggctgtgtatagatattctagagagggagatgatgcggaatcctatggcggtggaagatgccgtcacctcgcctttgttggcagatgatcttcacatgaagcttctctacctggaaaacagagttgcatttgctagattgttctttcctgctgaagccaaggtcgccatgcaaatcgcacaagcagacaccacagaagaattcggcggtatagttgcagttgcagcaagcacttctggtaaactgagggaggtggaccttaatgagacgccagtgacacaaaacaaaaggctccgttcaagggtagatgcactgatgaaaacagtggagctgggccgtcggtacttcccgaactgctcgcaggtgctggacaagttcctggaggacgatctgccggaaggtctggaccagttctacctccagaggggcacagccgatgagcagaaggtgaagaggatgcgcttctgcgagctgaaagaggacgtgctgaaggcgtttagcaaggacaaggcggagggcagcgtgttctcgggcctgtcctcgtcgtcgtcgtgctcgccgccccagaagtatgcccagaggtga

SEQ ID NO: 9 - DNA, 7789 bp, Zea mays
gcggccgcgtaatacgactcactatagggcgaagaattcggatctccttccttatttggcgaagccgaccgttggcgctttggagccgttggcgcaccggacactgtccggtgcacaccggacagtcaggtgcccccttccgaccgttggctcggccacgtgtttcgcgcggatcgcgcggcagaccgttggcccgaccgaccgttggctcaccggacagtccggtgcacaccagacagtccggtgaattatagccgtacgccgttaatcacttcccgagagcagcaagttcgcctgagccagcctggcgcaccggacactgtccggtgaaccaccggacagtccggtgcacccagtcagagctgactttggctgaacaaagtcatctttagttccaacttgatttttcctgtttccagcacttagacacaatacattagtctctaaaacaatgtattaattctgagaaacatacctttatacttggtttgtactttgtccaccatttaacacttgggcacttgtgttggacactaaatcaccaaaatacttagaaatggcccaagggcacatttccctttcaacagtccggtgccacaccggacagtccggtgacctctgacttctgtgttctaacttctgtcgcggcactgtttcgcactatagcgttttgcagtcgaccgttggcgcacagagagccattgctccgctggctgaccggacagtccgatgaattatagcggascgcgcctctgaattcccgagtgtggcctgtttgaagggcgcctggcctggtgcaccgaacaatgtatggtgcgccaaaaatcagcacactcaagtcctttgcttcattttttattgtgtcgctaactggatttctttttggtttgtgttgaaccttatgcacctgagataaatcacatctagccaaactagttagtccatgtggtttgtgttgatcgtcaactactaaaatctatttatagaaagtggttaaccctatttccctttcagcacactctatatagtgcttgagacctcgacatgaaggtgtcctaggaagccaaggctctcgcgtaaggtcctcgacatgcaggaccctaggccccgttagaatggggcttgtccataagagagttgggctctaagatgcatgactgacactgtgcgtctgtcgtttcttaataaagttatagatgatgttttgccaacatctgatgatatgtcttggtgcttacaaaagccttgttttttatcttcctttcgtcttaataaagatccatattacatttatatttactatgtcatatatatacctcactatctcgaagatacatctcgttgcggaagcataaggtagctttggaggtaaagcttagagcgacatgtgggtgcaacaaacaaacatgggggcacaacacacctcacctcatataactaatttggcttgcaaatcgagagtcccgtacgaaaagtactcgttgtctcttgacccaataaatcaaatacaccttcttacacaatttgtccattttatatttttcgtttccaataacaaactcaaagtgacttgtttttttggacctttgacacatagcctttaaagtagatttcacaatttaagcttgttatgtaaaacaaactaatttcgagagaggctgattgaggagaaagtctgcggtcgatgattcaattggacgaaatcgatgtttaaactgtcttgttgattaaatttctagcttcacacgtgcttgaacggcgtaggaagtgttggaatttcccttcttatgatttattagagtagagttttgttacagtttatttacggattcattacggtatttattagggatacgttgacatataacttcagtctttcttttttaatagtcacaagaaactttcacacacctactaggagtaacagaaaaacatggacatattgatttttgaaaaaagaaatattgacagataaggtgttggggaccgtagagactagagaggatgaggacgacgccaggcagacgagccttgccgattgccgtcgacgtcaccctggtcaggcgtcacttgacgacgtatacaggggcacagggctcaggttttccttcaaattgcgccgaaatactcgagatttcttggatttttttacttgtttattctattctccttccggcgcctctctagtctattctccttcctcgtcgagtcgtcgtcttcttcgatccactctttcccccatccctatctccctactttccacgcaactgcgtttcccccggactcttcttccacgattccgttggacccctaccgctcctcagtcagtcctcgcccctcccagcaccggccaacaatccctcacgttattccctgtagctactatgctgccctcttggatccctttttcacttgtctgagatttagccaccgcccggtaggaagaagaaggggaagcaccatattttctgttcctggcctgacgcagcgccggtgagatttcagtccgggatcggcaacgctgggaggactcgcgtgtgatttacgccgacttccgtgccgctctaggaagggtcacgtcgaggaggcttttgccgacgcggatttgcctggagccagccaagcagagcgcagaattgggggtgtttggccgtgcaaagccagaaagttctcggtttggctgccgaaaccgcttgaggcgaccaccatctcatggtcgtagtggtccgggtccggcacaagtaggggctcgcgtcttgcgcttggcagttgtgggaagccatggagccgtcgtcgtccatcacgttcgcgtcgtcgtcgtcgtacctgtccaacggctcgagcccctgttccgtcgcgctgccgccgccagggccgccccagactcccccgttgcctgccggccaggggtggggtggtggagtcgctgccgcagggagcggaggcagcgtggaggccgtgagcctgaaccggctcagcaaaaacctcgagcggctgctcctcgacccggacctagactgcagcgacgccgacgtcgatgtgcccgacggtgggccgcccgtacccatccaccgctgcatccttgccgcacgcagcgacttcttctacgacctcttcgccgctcgcggccgcgcaggggcagcgcgcggtgatgcggccgccggcgccggagtagccgcggagggggctgccagtggaaggccgcggtacaagatggaggatctcgttcccgccggccgcgtggggcgcgaggccttccaggcgtttctggggtacctgtacaccggcaagctccggccggcaccggtcgacgtggtgtcttgtgctgacccagtgtgccatcacgattcgtgcccgccggccatcaggtccgcggtcgagctcatgtacgcggcgtgtaccttcaagatccccgagctcacctcgctcttccaggtgagacgcaatttggttcttgctcgccccattgtcaataggattaaactctaatttctttaggaattgtttcgttctatgccaatactgtacatggcttcggtagactagaaatggatttgtgattttttttctccaatccgagtgttgactacatcactacaaaaagctatcaatagctgaactgctaaaattgctgattttgttttctccaatccgagtgttgaccacatcactgcaaaaagttattaatagctgaatcgctacaattgttcaattgttgattttgttttttccaatccgagtggtgacactacaaaaaactattaatagttgaactactaaaattgttggctattgcttttttagttgattcaagcgaacctggtggcttgaagtctatgaattgaaatggaatcattattcattaggctgctcaacatttgtatattacatttatggctgtataatttatcaatctgtttaacatcaattcagctttgctttgtcgatttatggaaggcaaatggttaacatggtcttcttctacagcgccggcttcttaattttgtagacaagactctagtggaggatgttattcctattctggaagttgcttcccactcagggctgactcaagtgatcgacaaatgtattcaaaggattgctagatcagatctcgacgatatatctttggataaggagctccctccagaagcagttgatgagataaaaaatttgcgcaagaagtcacaaactgctgatggtgatacgttcatttcggaccctgtgcatgagaaaagagtcagaagaatccacagggcacttgactctgatgatgttgagcttgtgaagttgcttcttaatgagtccgacatcacattagatgatgccaacgcattacactatgctgcttcttactgtgatcctaaagttgtctcagagctgttagatttggcaatggctaacttaaatttgaagaatagccgtgggtacacagcactccacttggctgctatgaggagagaaccagctataatcatgtgtctccttaacaaaggggcaaatgtgtcacaactgacagctgatggcaggagcgcaattggtatttgtcggaggttaacaagagcaaaagactacaatacaaagatggagcagggtcaagaatcaaataaagataggctgtgtatagatattctagagagggagatgatgcggaatcctatggcggtggaagatgccgtcacctcgcctttgttggcagatgatcttcacatgaagcttctctacctggaaaacagaggtgaagtccataccatgcttgatagaatggctctgattggttgcctgttgccgcttcaaattttgaaaatttaaaagcttggaggtcaggtggattgattcaggctagcttgtagactaatgacatgtgcctgaccttttgttctcataaagagggaaaaaggaaaacgcccaccccataccacatcaatttctcctttttttcaaattggtgaaagctgtacatgttgtaggaaataaacaattgtagtcacaaagcccaaattaatctaattacagatgacaagcctggattattaaattgccacttgcctgtccatattgcacacaacctagtagtgtcctagttctagataatataacgagaatgatttcacaccactggatgatgatcaaatagcaccttagaacttggggttgggatatgtcattgtcgtgagctttgtcttatgttcacgtttataagaagattgtgatttatgttattggctacattattttccctgcaccataacattctaagtattgttcctgcagttgcatttgctagattgttctttcctgctgaagccaaggtcgccatgcaaatcgcacaagcagacaccacagaagaattcggcggtatagttgcagttgcagcaagcacttctggtaaactgagggaggtggaccttaatgagacgccagtgacacaaaacaaaaggctccgttcaagggtagatgcactgatgaaaacaggtgaaagtttcaaccgtcaaccttttccattgtagtgaacatgccctgcagctatctccagaaaatttagttcggacgcaatcacggattctacgtgtacctgaagtattgtgactatgttcaaaatacatgattgatacaatgtatctgatctggtgttcgcccatttttatccgatgacttcttgcagtggagctgggccgtcggtacttcccgaactgctcgcaggtgctggacaagttcctggaggacgatctgccggaaggtctggaccagttctacctccagaggggcacagccgatgagcagaaggtgaagaggatgcgcttctgcgagctgaaagaggacgtgctgaaggcgtttagcaaggacaaggcggagggcagcgtgttctcgggcctgtcctcgtcgtcgtcgtgctcgccgccccagaagtatgcccagaggtgatcaaggcaccagtttttgccgtatagtttgttatcatggtcttcgagacttggacccggacagcatatagggacatgtacacctgtgtatgtatagtgcttacaattggcgtaagtagaactatatgtatggaacataaggaaacatggcaggaacaccgtgcaaaaagatgaaaagatggccgaagtgctctatgcgagtgcccacctgattcgatggccctattcaacggcgccctgtcagcatgctgcatgcccactgagaccttcggttgcatagggataggaggagatttctgttcaattttggctagcaagtgatataggggtgtttaggactgtttcactttatgaaaatcaacttagctcataaacacttttaacttcaataacttaggtcctgtttggagtggctgtatttttctagtcccaagaaaataatgtggtatctgagaataccatggtctagaaaccaaaatgtgtttggcagactcttttaaaccatggtatttaaaatcttggttttgacaagaccacattatttctgggtatagaatactgtccagactagggctggaaatgagccgagttcggctcggctcggtgcggctcgttgactgaacgagctcgactcggctcgtccattccacgagctggtgaaagaggctcggctcggctcgaccttagctcgcgagccatattcttgtacttatcgtttcattatttgatgaattaacattatataaatttgaaatataaaatcatcattctacattatgagtaaattaaactataacttgtaatatattcatcatcaaagactaaaaaataagctattatccataaaattatctaatatttattattattccataaattgatcatttttgcaaggctcgtgagctggaacgagccggctcggctcggctcgctgcaaaaacgagctcgaagaaggggctcggcttggctcgttcgaggctcgcgagctgcgccgagccgagccgctccgagctcgagccggctcgcgagcctcgagctaattttccagccctagtccagaccaaagtttttcgtcagagcgcgcgaagcgaccctaatgtccgcgcccttttctcaccgtccacgcatctcttcccctcaattctaaggctttctccaacaagaaacgttaaagcgttcggtacgctatatttagcgtgtaccttcgtctccaacaagcacatgtatagcgttcgctaaaatttagcggagcctcagcgtccgccaaatctagcatctcctgtccgtccgctattctgtcagctctcgtacgggaagccgtatgctactatcatagcgccaataaaaaacaaacaatgccaggaacgtctccgctgaccagtaaaaaagaactaaaaatggaactaaaagatcttctatggacatatgtgagaacgaccatttcatatattcaacatcttttagctgtacataaatattttactatgtattctacaataatttgtaatttgttgaaaaatatgattgcaataaattatgttaatatggttgccaaaatatatacgatgagatatagaacaaacattgtagtttatggattatgttaacactgtagatatagagattcgaatttaggtgacgttgctgaagatgaagaagatatagagaacataatcttttagagaatgctgtaaaggacagagaatatttctttagagaacggaatttagggtacgttgctggagacagcctaatacagacactcttttcctctttctccattccccatcgcaaaactgggaggacctagcttcgccgcagatgtcgtcggtcccgggacgctgatctcgcctcagattccgtgg

SEQ ID NO: 10 - DNA, 24206 bp, Klebsiella pneumoniae
  exon (260)..(3775)
  exon (4178)..(5059)
  exon (5073)..(6524)
  exon (6580)..(8142)
  exon (8182)..(8400)
  exon (8411)..(9073)
  exon (9395)..(10768)
  exon (10779)..(12164)
  exon (12151)..(12621)
  exon (12817)..(13641)
  exon (13672)..(14874)
  exon (14889)..(16031)
  exon (16031)..(16291)
  exon (16288)..(16734)
  exon (16731)..(17531)
  exon (17555)..(18085)
  exon (18407)..(19894)
  exon (19891)..(21465)
  exon (21630)..(23036)
  exon (23036)..(23539)
ggtaacccgctacggcttgagattatccgcatccttgccgacggcagcgagcagagctgtaacgccctgcgtcacgaagatgtggcgaagtcgaccatgacccaccactggcgcgtcctgcgcgacagcggtgtgatctggcagcgcccacaggggcgggagaacttgatttcgctgcgccgggaagatttagacgcgcgctttcccggcctgctggatacgctgcttaaggtcatgcagcaggagaactaaaggcccgctactcctcgccggccagccgccgatactgggcaaagcgggcccgcgcgtcctcctcggttcggctaaagagcgcatccgccagatgcggcgtcgttttgtgcagcgaggcgtagcgcacttcgccaagcaaaaagtcgcggaagctctcctccggctcttcggaatcgagcataaacggcgtcttaccttccgcttcccgctgcggatgatagcgccacaggtgccagtatcccgcctcaaccgcccgtttcgcctcgcgctggctgcagcgcataccggctttcagcccgtggttaatgcaggcggcgtaggcaatcaccagcgacggtcccggccaggcttcggcctcggcgatcgcccgtagggtctgatctttatcagcgcccatcgcgacctgggccacgtacacattgccgtagctcatcgccatcatgccgagatcttttttccgcgtgcgtttgccctgcgcggcaaacttcgcgatggccgccaccggggtcgatttagacgactggccgccggtattggagtaaacctcggtgtcaaacaccagaatattgacgtcttccccgctcgccagcacgtgatcgagaccgccgaagccgatatcgtaggcccagccgtcgccgccgaaaatccactgcgaacgacgaacaaaatagtcgcggttctgccacagctgctccaacagcggcacgccctctttttccgccgccagccgttcgctgagccggtccgcgcgctcgcgggtgccctcgccttcatcctgcttcgccagccactggcgcattgcgtcgctaagttcgtcgctgaccggtagcgccagcgcggcggtcatatcatcggcgatttgttgacgcaccgcctggccgccgagcatcatgccgaggccaaactccgcattatcctcaaacagcgagttcgcccatgccgggccatggccgcggtggttggtggtatagggaatcgacggcgcgctggctccccagatagaagagcagccggtggcgttagcgatcagcatccggtcgccaaacagctgggttatcaggcgggcataaggcgtttcaccgcatcccgcgcaggcgccggaaaactccagcagcggggtttcaaactggctgcctttgaccgtcgtcttacgaaacggattgctcttcggcgtcagcgccagcgcatagtcccagaccggcgccatctgacgctggctatcgagagactgcatttttaacgccttgccgcgcgcgggacagatatccacgcagttgccgcagccggaacaatccagcggcgagatagccagatggtagtgatactccttcgctccctgcgcgggtttgctcagcagcccaaccggcgcggcgtcatgctcttcgccgttgagcagcgccgggcggatcgccgcatgcgggcagataaaggcgcactggttacactgcgtgcagccctccggctgccagaccggcacttccagcgcgatcccgcgtttctcccacgcggcggtgcccgaaggaaaggtcccgtcctccataccgacgaacgcgctcaccggcagctggtcgccgcactggcggttcatcggctgcagaatatcgcggatgaaatccggcatcatggctgatgcttgcgccgcgggttcatccagcgtcgcccagtgcgccggaatcgtcacctgatgcagcgaggccatgcccagctcgatcgcccgctggttcatctcaatcaccgccgcccctttgctgccgtagcttttttcaaccgcctgcttgaggtaatccgccgcggtctgcgggtcgataatcgccgccagcttaaagaacgccgcctgcatcagcatattaaagcgcccgcccagcccgagctcgcgggcgatatccacggcgttcagggtataaaaatggatattttcccgcgccagatagcgtttaaagccgaccggcagatgctgctccagctccgcatcggaccagctgcagttgagtaaaaaggtcccgcccggctttaatccgtccagcagatcgtagcgctcaacgtaggactgctgcgaacaggagataaaatcggcccgatggatcaggtagggcgaattgatcggccggtcgccgaagcgtaaatgtgaaacggtaatgccgccggattttttcgagtcataagaaaagtaggcctgcgcgtagagcggcgttttatcgccgataattttgatcgcgcttttattggccccgacggtgccgtccgagcccatgccccaaaatttacaggcggtgatgccgtcatgcgagaccgccagcgtctgctgggccggcggtaacgaagtaaaggttacatcatcgacaatcccgagggtaaacccgtccatcggcagcggtttattgaggttatcaaagacggccgcgatatcgttgggcagaacatccttcccgccaagcgcatagcggccgccgacgattagcggcgcatcgtcgtggtggtagaaggcgtttttcacatccaggcacagcggttcagcctgagcgccgggctctttggtacggtcaaggacggcaatccgctgcacggttttcggcagctgggcgaagaagtgggccagcgaaaaagggcgaaacagatgcacgctgagcagcccgaccttctctcccgccgcgttcagcgtatccaccacttcctgaacggtatcgcagaccgatcccattgcgataatcacccgttcggcatccgccgcgccggtatagttaaacagatgatactcccggccggtgagcgcgctgatttgcgtcatatagctttcgacaatgtcgggcagcgcctgataaaaacggttgcccgcctcccgctcctggaagtagatatccgggttctgcgccgttccgcggatgaccggatgatccggatgcagcgcgttacggcggaagctgtcgagcgcgggccggtccagcagcgtcgccagctgctcatattccaacacctcgattttttgaatttcgtgcgaggtgcgaaaaccgtcgaagaagttaacaaacgggatgcgtcccttaatcgccgccagatgcgccaccgccgacaaatccatcacctgctgcacgttgttctccgccagcatcgcgcagccggtctggcggaccgccatcacatcctggtgatcgccaaaaatattcagcgaattggtcgccagcgcccgggcgctgacgtgaaagacgcccggcagcagttcaccggcgattttgtacatgttggggatcatcagcagcagcccctgggaggccgtataggtggtggtgagcgccccggcctgcagcgcgccgtggaccgcgcctgccgcgccggcctccgactgcatctccattaagcgcaccggctggccaaaaaggttctttttcccctgcgccgcccactcgtcgacgttttccgccatcggcgtggagggggttatggggtaaatcgccgcgacctcggtaaaggcataagagatccaggccgccgcggcgttgccatccattgttttcatttttccggacattgttcaatcctcgaaggtgagaggcatcttcgccgcctcaaataagcggcaaacccagttgttgcctcaagcacagcctgtgccagctcgcggatgacagaagagttagcgcgaattcaacgcgttatgaagagagtcgccgcgcagcgcgccaagagattgcgtggaataagacacagggggcgacaagctgttgaacaggcgacaaagcgcccatggccccggcaggcgcaattgttctgtttcccacatttggtcgccttattgtgccgttttgttttacgtcctgcgcggcgacaaataactaacttcataaaaatcataagaatacataaacaggcacggctggtatgttccctgcacttctctgctggcaaacactcaacaacaggagaagtcaccatgaccatgcgtcaatgcgctatttacggtaaaggcggtatcggtaaatccaccaccacgcagaacctcgtcgccgcgctggcggagatgggtaagaaagtgatgatcgtcggctgcgatccgaaggcggactccacccgtctgattctgcacgccaaagcacagaacaccattatggagatggccgcggaagtcggctcggtcgaggacctcgaactcgaagacgtgctgcaaattggctacggcgatgtgcgctgcgcggaatccggcggcccggagccaggcgtcggctgcgcgggacgcggcgtgatcacggcgatcaactttcttgaagaagaaggcgcctacgaggacgatctcgatttcgtgttctatgacgtgctcggcgacgtggtctgcggcggcttcgccatgccgatccgcgaaaacaaagcccaggagatctacatcgtctgctccggcgaaatgatggcgatgtacgcggccaacaatatctccaaagggatcgttaaatacgccaaatccggcaaggtgcgcctcggcggcctgatctgtaactcacgtcagaccgaccgtgaagacgaactgattattgccctggcggaaaagctcggtacccagatgatccactttgtgccccgcgacaacatcgtgcagcgcgcggagatccgccgcatgacggttatcgagtacgaccccgcctgtaaacaggccaacgaataccgcaccctggcgcagaagatcgtcaacaacaccatgaaagtggtgccgacgccctgcaccatggatgagctggaatcgctgctgatggagttcggcatcatggaagaggaagacaccagcatcattggcaaaaccgccgccgaagaaaacgcggcctgagcacaggacaattatgatgaccaacgcaacgggcgaacgtaatctggcgctgatccaggaagtcctggaggtgttcccggaaaccgcgcgaaaagagcgcagaaagcacatgatggtcagcgatccgaaaatgaagagcgtcggcaagtgcattatctctaaccgcaaatcacaacccggcgtaatgaccgtacgcggctgcgcctacgccggttccaaaggggtggtatttgggccgattaaggatatggcccatatttcgcacggaccggctggctgcggccagtattcccgcgccgaacgacgcaactactacaccggagtcagcggcgtcgatagcttcggcacgctgaacttcacctctgattttcaggagcgcgacatcgtcttcggcggcgataaaaagctcagcaagctgattgaagagatggagttgctgttcccgctcaccaaagggatcaccattcagtcggaatgcccggtggggctgatcggtgatgatatcagcgcggtggccaacgccagcagcaaggcgctggataaaccggtgatcccggtacgctgcgaaggctttcgcggcgtgtcgcagtctctggggcaccatatcgccaacgacgtggtgcgcgactggatcctgaacaatcgcgaaggacagccgtttgaaaccaccccttacgatgtggcgatcatcggcgactacaacatcggcggcgacgcctgggcctcgcgcattctgctggaagagatggggctacgggtagtcgcgcagtggtccggcgacggcacgctggtggagatggagaataccccattcgtcaagctgaacctggttcactgctaccgttcgatgaactatatcgcccgccatatggaggagaaacatcagattccgtggatggagtacaacttcttcgggccgaccaaaatcgccgaatcgctgcgcaaaatcgccgaccagttcgacgataccattcgcgcgaacgccgaagcggtgatcgcccggtatgaggggcagatggcggcgattatcgccaaatatcgcccgcgcctggaggggcgtaaggtgctgctctatatcggaggcctgcggccgcgccacgttattggcgcctatgaggatctcgggatggagatcatcgccgccggctacgagtttgcccataacgatgattacgaccgcaccctgccggatctgaaagagggcacgctgctgttcgatgacgccagcagctacgagctggaagcgttcgtcaaggcgctgaagcccgaccttatcggctccggcatcaaggaaaaatatatcttccagaaaatgggcgtgccgttccgccagatgcactcgtgggactattccggcccgtaccacggctacgatggtttcgccattttcgcccgcgatatggatatgaccctgaacaacccggcgtggaacgaactgaccgctccgtggctgaagtctgcgtgattgcccactcactgtcccgtctgttcaccgatttgtggcgcgggaggagaacaccatgagccaaacgattgataaaattaatagctgttatccgctattcgaacaggatgaataccaggagctgttccgcaataagcggcagctggaagaggcgcacgatgcgcagcgcgtgcaggaggtctttgcctggaccaccaccgccgagtatgaagcgctgaatttccgacgcgaggcgctgaccgttgacccggcgaaagcctgccagccgcttggcgcggtgctttgctcgctgggatttgccaacaccctgccgtatgtgcacggctctcaggggtgcgtggcctactttcgcacctattttaaccgccatttcaaagagccgatcgcctgcgtctccgactcgatgaccgaagacgcggcggtcttcggcggcaacaacaatatgaacctgggcctgcagaacgccagcgcgctgtacaaaccggagatcattgcggtgtccaccacctgcatggcggaagttatcggcgatgacctgcaggcgtttatcgccaacgctaaaaaagatggcttcgtcgacagcagcatcgccgtgccccacgcccatacgccaagctttatcggcagccacgtcaccggctgggataacatgtttgaaggcttcgccaaaaccttcactgcggactaccaggggcagccgggcaaattgccgaagctcaatctggtgaccggctttgaaacctatctcggcaacttccgcgtattaaagcggatgatggaacagatggcggtgccgtgcagcctgctctccgatccgtcggaagttctcgacacgcccgccgacggtcactatcggatgtattccggcggcaccacgcagcaggagatgaaagaggcccctgacgccatcgatacgctgctcctgcagccgtggcagctgctgaagagcaaaaaagtggtgcaggagatgtggaaccagcccgccaccgaggtcgccattccgctggggctggccgccaccgatgaactgctgatgaccgtcagccagcttagcggcaagccgattgccgacgccctcacccttgagcgcggccggctggttgacatgatgctcgactcccacacctggctgcacggcaagaagtttggcctgtacggcgatccggacttcgtgatgggcctcacccgcttcctgctggagctgggctgcgagccaacggtgatcctgagccataacgccaacaaacgctggcaaaaagcgatgaacaaaatgctcgatgcctcgccgtacgggcgcgatagcgaagtgtttatcaactgcgatttgtggcacttccgttcgctgatgttcacccgtcagccggactttatgatcggcaactcctacggcaagtttatccagcgcgataccctggcgaagggtaaagcctttgaagtgccgcttatccgcctcggctttccgctgttcgaccgccaccatctgcaccgccagacaacctggggttatgaaggggcgatgaacattgtgacgacgctggtgaacgccgtgctggagaaactggatagcgataccagccagctgggcaaaaccgattacagcttcgatctcgtccgttaaccatcaggtgccccgcgtcatgcggcggcaggagggagtatgcccatcgtgattttccgtgagcgcggcgcggacctgtacgcctatatcgcgaaacaggatctggaagcgcgagtgatccagattgagcataacgacgctgaacgctggggcggcgcgatttcgctggaggggggacgccgctactacgtgcatccgcagccggggcgtcccgtctttccgataagcctgcgcgcgacgcgcaataccttgatataaggagctagtgatgtccgacaacgataccctattctggcgtatgctggcgctgtttcagtctctgccggacctacagccggcgcaaatcgtcgactggctggcgcaggagagcggcgagacgctgacgccagagcgtctggcgaccctgacccagccgcagctggccgccagctttccctccgcgacggcggtgatgtcccccgctcgctggtcgcgggtgatggcgagcctgcagggcgcgctgcccgcccatttacgcatcgttcgccctgcccagcgcacgccgcagctgctggcggcattttgctcccaggatgggctggtgattaacggccatttcggccagggacgactgttttttatctacgcgttcgatgaacaaggcggctggttgtacgatctgcgccgctatccctccgccccccaccagcaggaggccaacgaagtgcgcgcccggcttattgaggactgtcagctgctgttttgccaggagataggcgggcccgccgccgcgcggccgatccgccatcgcatccacccgatgaaagcgcagcccgggacgacgattcaggcacagtgcgaggcgatcaatacgctgctggccggccgtttgccgccgtggctggcgaagcggcttaacagggataaccctctggaagaacgcgttttttaatccctgttttgtgcttgttgcccgctgaccccgcgggctttttttcgcgtatggacgctcttccccacgttacgctcaggggaatattccgttcacggttgttccgggcttcttgatgcgcctaaccccctcgctgccagcctttcatcaacaaatagccatcccagcgcgataggtcataaagcatcacatgccgccatcccttgtccgattgttggctttgtcgcaaagccaacaacctcttttctttaaaaatcaaggctccgctctggagcgcgaattgcatcttccccctcatcccccaccgtcaacgaggtcactatgaagggaaatgaaattctggcgctgctggatgaaccggcctgtgaacacaaccataaacaaaaatccggctgcagcgcgcccaaacccggcgccaccgccgcgggctgcgcgttcgacggcgcgcagataaccctgctgcccatcgccgacgtggcgcatctggtccacggccccatcggctgcgccggaagctcatgggataaccgcggcagcgccagctccggccccacccttaatcggctcgggttcaccaccgatctcaacgaacaggacgtgattatgggccgcggcgaacgccgactgtttcacgccgtgcgccatatcgtcacccgctatcatccggcggcggtctttatctacaacacctgcgtaccggccatggagggcgatgacctggaagcggtatgccaggccgcgcagaccgccaccggcgtaccggttatcgctattgacgccgccggtttctacggcagtaaaaatctcggtaaccggccggcgggcgacgtcatggtcaaacgggtcatcggccagcgcgagcccgccccctggccggagagcacgctctttgccccggagcagcgtcacgatattggcctgattggcgaattcaatattgccggcgagttctggcatattcagccgctgctcgacgaactggggatccgcgtgctcggcagcctctccggtgatggccgcttcgccgagatccagaccatgcaccgggcgcaggccaatatgctggtctgctcgcgggcgttaattaacgtcgccagagccctggagcagcgctacggcacgccgtggttcgaaggcagcttttacgggatccgcgccacctctgacgccctgcgccagctggcggcgctgctgggcgacgacgaccttcgccagcgcaccgaagcgctgattgcgcgggaggaacaggcggcggaactggcgctacagccgtggcgcgaacagctgcgcggccgcaaagcgctgctctataccggcggggtgaaatcctggtcggtggtatcggcgctgcaggatttgggcatgaccgtggtggcaaccggcacgcgtaaatccaccgaagaggataaacagcggatccgcgagctgatgggcgaagaggcggtaatgctggaagagggcaacgcccgcacgctgctggatgtggtctatcgctatcaggccgacctgatgattgccggcggacgcaatatgtacaccgcctataaagccaggctgccgtttctcgatatcaatcaggagcgcgaacacgccttcgctggctatcaggggatcgtcaccctcgcccgccagctgtgtcagaccatcaacagccccatctggccgcaaacccattctcgcgccccgtggcgctaaggagctcaccatggcagacattttccgcaccgataagccgctggcggtcagccccatcaaaaccggccagccgctcggcgcaatcctcgccagcctcgggatcgaacacagcatccctctggtccacggcgcgcaggggtgcagcgccttcgccaaagtcttttttattcaacatttccacgacccggttcccctgcagtcgacggcgatggaccccacgtcgacgattatgggcgcggacggcaatatttttaccgccctggataccctctgccagcgcaacaatccgcaggctatcgtactgctcagcaccgggctgtcggaggcccagggcagcgatatttcccgcgtggttcgccagtttcgcgaagagtatccccggcataagggggtggcgatattgacggttaacacgccggatttttatggctccatggagaacggcttcagcgcggtgttagagagcgtcattgagcagtgggtgccgccggcgccgcgcccggctcagcgcaatcgccgggtcaatctgctggtcagccatctctgttcgccgggcgatatcgagtggctgcgccgatgcgtcgaagcctttggtctgcagccgataatcctgccggacctggcgcaatcgatggacggccacctggcgcagggcgatttctcgccgctgacccagggcgggacgccgctgcgccagatagagcagatggggcaaagcctgtgcagcttcgccattggcgtctcccttcatcgcgcctcatcgctgctggccccgcgctgccgcggcgaggttatcgccctgccgcacctgatgaccctcgaacgctgcgacgcctttattcatcaactggcgaaaatttccggacgcgccgttcccgagtggctggaacgccagcgcggccagctacaggatgcgatgatcgactgccatatgtggctccagggccagcgcatggcgatagcggcggaaggcgatttgctggcggcgtggtgtgatttcgccaacagccaggggatgcagcccggcccgctggtggcccctaccggtcatcccagcctgcgccagctgccggtggaacgggtggtgccgggggatctggaggatctgcaaaccctgctgtgcgcgcatcccgccgacctgctggtggcgaactcgcacgcccgcgacctggcggagcagtttgcgctgccgctggtgcgcgcgggttttccgctctttgacaagctcggcgaattccgccgggtgcgacaggggtatagcgggatgcgcgatacgctgtttgagctggcaaacctgatacgcgagcgtcaccaccacctcgcccactaccgatcgccgctgcgccagaaccccgaatcgtcactctccacaggaggcgcttatgccgccgattaaccgtcagtttgatatggtccactccgatgagtggtctatgaaggtcgccttcgccagctccgactatcgtcacgtcgatcagcacttcggcgctaccccgcggctggtggtgtacggcgtcaaggcggatcgggtcactctcatccgggtggttgatttctcggtcgagaacggccaccagacggagaagatcgccaggcggatccacgccctggaggattgcgtcacgctgttctgcgtggcgattggcgacgcggtttttcgccagctgttgcaggtgggcgtgcgtgccgaacgcgttcccgccgacaccaccatcgtcggcttactgcaggagattcagctctactggtacgacaaagggcagcgcaaaaatcagcgccagcgcgacccggagcgctttacccgtctgctgcaggagcaggagtggcatggggatccggacccgcgccgctagccgtgtcgtttctgtgacaaagcccacaaaacatcgcgacactgtaggacgaaccttgtcaggactaatacacaaccatttgaaaaatattaattttattctctggtatcgcaattgctagttcgttatcgccaccgcgcttccgcggtgaaccgcgccccggcgttttccgtcaacatccctggagctgacagcatgtggaattactccgagaaagtgaaagaccatttttttaacccccgcaatgcgcgcgtggtggacaacgccaacgcggtaggcgacgtcggttcgttaagctgcggcgacgccctgcgcctgatgctgcgcgtcgacccgcaaagcgaaatcattgaggaggcgggcttccagaccttcggctgcggcagcgccatcgcctcctcctccgcgctgacggagctgattatcggccataccctcgccgaagccgggcagataaccaatcagcagattgccgattatctcgacggactgccgccggagaaaatgcactgctcggtgatgggccaggaggccctgcgcgcggccatcgccaactttcgcggcgaaagccttgaagaggagcacgacgagggcaagctgatctgcaaatgcttcggcgtcgatgaagggcatattcgccgcgcggtacagaacaacgggctgaccacccttgccgaggtgatcaactacaccaaagcgggcggcggctgcacctcttgccacgaaaaaatcgagctggccctggcggagatcctcgcccagcagccgcagacgacgccagccgtggccagcggcaaagatccgcactggcagagcgtcgtcgataccatcgcagaactgcggccgcatattcaggccgacggcggcgatatggcgctactcagcgtcaccaaccaccaggtgaccgtcagcctctccggcagctgtagcggctgcatgatgaccgatatgaccctggcctggctgcagcaaaaactgatggaacgtaccggctgttatatggaagtggtggcggcctgagccggcgttaactgacccaagggggacaagatgaaacaggtttatctcgataacaacgccaccacccgtctggacccgatggtcctggaagcgatgatgccctttttgaccgatttttacggcaacccctcgtcgatacacgattttggcattccggcccaggcggctctggaacgcgcgcatcagcaggctgcggcgctgctgggcgcggagtatcccagcgagatcatctttacctcctgcgccaccgaagccaccgccaccgccatcgcctcggcgatcgccctgctgcctgagcgtcgcgaaatcatcaccagcgtggtcgaacatccggcgacgctggcggcctgcgagcacatggagcgcgagggctaccggattcatcgcatcgcggtagatggcgagggggcgctggacatggcgcagttccgcgcggcgctcagcccgcgcgtcgcgttggtcagcgtgatgtgggcgaataacgaaaccggggtgcttttcccgatcggcgaaatggcggagctggcccatgaacaaggggcgctgtttcactgcgatgcggtgcaggtggtcgggaaaataccgatcgccgtgggccagacccgcatcgatatgctctcctgctcggcgcataagttccacgggccaaaaggcgtaggctgtctttatctgcggcggggaacgcgctttcgcccgctgctgcgcggcggtcaccaggagtacggtcggcgagccgggacagaaaatatctgcggaatcgtcggcatgggcgcggcctgcgagctggcgaatattcatctgccgggaatgacgcatatcggccaattgcgcaacaggctggagcatcgcctgctggccagcgtgccgtcggtcatggtgatgggcggcggccagccggcggtgcccggcacggtgaatctggcctttgagtttattgaaggtgaagccattctgctgctgttaaaccaggccgggatcgccgcctccagcggcagcgcctgcacctcaggctcgctggaaccctcccacgtgatgcgggcgatgaatatcccctacaccgccgcccacggcaccatccgcttttctctctcgcgctacacccgggagaaagagatcgattacgtcgtcgccacgctgccgccgattatcgaccggctgcgcgcgctgtcgccctactggcagaacggcaagccgcgcccggcggacgccgtattcacgccggtttacggctaaggcggaggtggctgatggaacgcgtgctgattaacgataccaccctgcgcgacggcgagcagagccccggcgtcgcctttcgcaccagcgaaaaggtcgccattgccgaggcgctttacgccgcaggaataacggcgatggaggtcggcaccccggcgatgggcgacgaggagatcgcgcggatccagctggtgcgtcgccagctgcccgacgcgaccctgatgacctggtgtcggatgaacgcgctggagatccgccagagcgccgatctgggcatcgactgggtggatatctcgattccggcttcggataagctgcggcagtacaaactgcgcgagccgctggcggtgctgctggagcggctggcgatgtttatccatcttgcgcataccctcggcctgaaggtatgcatcggctgcgaggacgcctcgcgggccagcggccagaccctgcgcgctatcgccgaggtcgcgcagcaatgcgccgccgcccgcctgcgctatgccgatacggtcggcctgctcgacccttttaccaccgcggcgcaaatctcggccctgcgcgacgtctggtccggcgaaatcgaaatgcatgcccataacgatctgggtatggcgaccgccaatacgctggcggcggtaagcgccggggccaccagcgtgaatacgacggtcctcggtctcggcgagcgggcgggcaacgcggcgctggaaaccgtcgcgctgggccttgaacgctgcctgggcgtggagaccggcgtgcatttttcggcgctgcccgcgtcctgtcagagggtcgcggaagccgcgcagcgcgccatcgacccgcagcagccgctggtcggcgagctggtgtttacccatgagtcaggtgtccacgtggcggcgctgctgcggcacagcgagagctaccagtccatcgccccttccctgatgggccgcagctaccggctggtgctgggcaaacactccgggcgtcaggcggtcaacggcgtttttgaccagatgggctatcacctcaacgccgcgcagattaaccagctgctgcccgccatccgccgcttcgccgagaactggaagcgcagcccgaaagattacgagctggtggctatctacgacgagctgtgcggtgaatccgctctgcgggcgagggggtaatgatggagtggttttatcaaattcccggcgtggacgaacttcgctccgccgaatctttttttcagtttttcgccgtcccctatcagcccgagctgcttggccgctgcagcctgccggtgctggcaacgtttcatcgcaaactccgcgcggaggtgccgctgcaaaaccggctcgaggataacgaccgcgcgccctggctgctggcgcgaagactgctcgcggagagctatcagcaacagtttcaggagagcggaacatgagaccgaaattcacctttagcgaagaggtccgcgtcgtacgcgcgattcgtaacgacggcaccgtggcgggcttcgcgcccggcgcgctgctggtcaggcgcggcagcaccggctttgtgcgcgactggggcgtttttttgcaagatcagattatctaccagatccactttccggaaaccgatcggatcatcggctgccgcgagcaggagctgatccccatcacccagccgtggctggccggaaatttgcaatacagggatagcgtgacctgccagatggcgctcgcggtcaacggcgatgtggtcgtgagcgccggccagcggggacgcgttgaggctaccgatcggggagagctcggcgacagctacaccgtcgactttagcggccgctggttcagggtcccggtgcaggccatcgcccttatagaggaaagagaagaatgaacccgtggcaacgttttgcccggcagcggctggcgcgcagccgctggaatcgcgatccggcggccctggatccggccgacacgccggcttttgaacaggcctggcaacgccagtgccatatggagcagacgatcgtcgcgcgggtccctgaaggcgatattccggcggcgttgctggagaatatcgctgcctcccttgccatctggctcgacgagggggattttgcgccgcccgagcgcgctgccatcgtgcgccatcacgcccggctggaactcgccttcgccgatatcgcccgccaggcgccgcagccggatctctccacggtacaggcatggtatctgcgccaccagacgcagtttatgcgcccggaacagcgtctgacccgccatttactgctgacggtcgataacgaccgcgaagccgtgcaccagcggatcctcggcctgtatcggcaaatcaacgcctcgcgggacgctttcgcgccgctggcccagcgccattcccactgcccgagcgcgctggaagagggtcgtttaggctggattagccgtggcctgctctatccgcagctcgagaccgcgctgttttcactggcggaaaacgcgctaagccttcccatcgccagcgaactgggctggcatcttttatggtgcgaagcgattcgccccgccgcgcccatggagccgcagcaggcgctggagagcgcgcgcgattatctttggcagcagagccagcagcgccatcagcgccagtggctggaacagatgatttcccgtcagccgggactgtgcgggtagcctcggcggctacccgttaacgcctacagcacggtgcgtttaatctcctcaagccagctcgccagacgcgcttcggtctggtcgaactggttatcctgatccagcaccagcccaacaaagcggtcgccttccagcgccgaggacgcgctgaattcataaccctcatttggccagctgccaatcatctgcgcgccgcgcgcgctcagggcgtcgaacagcgggcgcatcccgctgacgaagttgtccggatagcctctctgatcgccgaggccgaacagcgccacggttttccctttcaggctggcgtcgtcgaggccgctgataaattcgctccatgactcgctttcgcatccggcctccagccccggcagctggccgtcgccgagcgtcggcgtgcccagcagcagcaccggataggccataaagtcgtccagcgtcgtgcggttaatgttgaccggggcatccgccagctcgcccagttgcttatggatcattttcgcgattttgcgggttttaccggtatcggtgccaaagaaaataccaatgttcgccatgttgcgctcctgtcggaaaagggggttgaaaatacgcgttctcgcaggggtattgcgaaggctgtgccaggttgctttgcactaccgcggcccatccctgccccaaaacgatcgcttcagccctctcccgccgcgcgcggcggggctggcggggcgcttaaaatgcaaaaagcgcctgcttttcccctaccggatcaatgtttctgcacatcacgccgataagggcgcacggtttgcatggttatcaccgttcggaaaacaccgcggcgtccctgtcacggtgtcggacaaattgtcataactgcgacacaggagtttgcgatgaccctgaatatgatgctcgataacgccgtacccgaggcgattgccggtgcgctgactcaacaacatccggggctgttttttacaatggtcgaacaggcatcggtagcgatttccctcaccgatgcccgggcgaatattacctacgccaacccggcgttttgccgccagactggatactcgctggcgcaattgctcaatcaaaacccgcgcctgctggccagcagccagacgccgcgcgagatctaccaggagatgtggcaaaccctgctccagcgccagccgtggcgcggtcagctaattaatcaggcccgcgacggcggcctgtatctggtagatatcgatatcacgccggtgctgaatccgcagggcgagctggagcattatctggcgatgcagcgggatatcagcgtcagctataccctggaacagcggctgcgcaatcatatgacgctaatggaagcggtgctcaataacatccccgccgccgtggtcgtggtcgatgagcaggatcgggtggtgatggataatctcgcctacaaaacgttctgcgcggactgcggcgggaaagagctgctggtcgagctccaggtttccccgcgcaaaatggggcccggcgcggagcaaatcctgccggtggtggttcgcggcgcggtccgctggctgtcggtaacctgctgggcgctgcccggcgtgagtgaagaagccagccgctacttcgtcgacagcgccccggcgcgcacgctgatggtgatcgccgactgtacccagcagcgccagcagcaggagcagggccggctcgaccgtctgaaacagcaaatgaccgccggtaagctgctggccgcgattcgcgagtcgctggacgcggcgctgattcagcttaattgcccaatcaatatgctggcggcggcccgccggctgaacggcgaaggcagcggcaacgtggcgctggacgcggcgtggcgcgaaggtgaagaggccatggcgcgcctgcagcgctgccgcccttctcttgagctggaaagcaatgccgtctggccgcttcagcccttttttgacgacctgtacgccctctaccgcacccgctttgacgatcgcgcgcggctgcaggtggacatggcatcgccgcatctggtcggcttcggccagcgtacccagctgctggcctgcttgagtttatggctcgaccggacgctggccctcgccgccgagctgccctccgtaccgctggagatcgagctttacgccgaagaggacgagggctggctctctttgtatctcaacgacaatgtcccgctgctgcaggtgcgctacgcccactcccccgatgccctaaactctcccggcaaagggatggagctgcggctgatccaaacgctggtcgcctaccaccgcggcgcgattgaactggcttcgcgaccgcagggaggcaccagcctggttctgcgtttcccgctctttaataccctgaccggaggtgagcaatgatccataaatccgattcggacaccaccgtcagacgtttcgatctctcccagcagtttaccgccatgcagcggataagcgtggtcctgagtcgcgccaccgaagcgagcaaaaccctgcaggaggttctgagcgtgctacataacgatgcctttatgcagcacgggatgatttgcctgtacgacagccagcaggagatcctgagcatcgaagcgctgcagcaaacggaagatcagacgctgcccggcagtacgcaaattcgctaccggccgggggaaggattagtcggtaccgtgctggcgcagggccagtcgctggtgctgccgcgcgtcgccgacgaccagcgttttctcgatcgtctgagcctgtacgactatgacctgccgtttatcgccgttccgctgatgggcccccactcccggcccatcggcgtactggcggcgcacgcgatggcgcgtcaggaagagcggctgcccgcctgcacgcgctttctcgaaaccgtcgccaatctgatcgcccagacgattcgcctgatgatcctgccaacctccgccgcgcaggcgccgcagcagagccccagaatagagcgcccgcgcgcctgtaccccttcgcgcggtttcggcctggaaaatatggtcggtaaaagcccggcgatgcggcagattatggatattattcgtcaggtttcccgctgggataccacggtgctggtacgcggcgagagcggcaccgggaaagagctcatcgccaacgccatccaccataattctccgcgcgccgccgcggcgttcgtcaaatttaactgcgcggcgctgccggacaacctgctggagagcgagctgtttggtcatgagaaaggcgcgtttaccggcgcggtgcgccagcggaaaggccgctttgagctggcggacggcggcaccttattcctcgatgagatcggcgaaagcagcgcctcgtttcaggctaagctactgcgtattctgcaagagggggagatggagcgcgtcggcggcgacgaaaccctgcgggtcaacgtgcgcattatcgcggcgaccaaccgccatctggaagaggaggtgcggctgggtcatttccgcgaggatctatactaccgcctgaacgtaatgcctatcgcgctgccgccgctgcgcgagcgccaggaggatatcgccgagctggcgcactttctggtgcgaaaaatcgcccacagccaggggcgaacgctgcgcatcagcgatggggcgattcgcctgctgatggagtacagctggccgggaaacgtgcgcgaactggaaaactgtctcgaacgttcggcggtgctgtcggaaagcggcctgatagaccgggacgtgattctgttcaaccatcgcgataacccgccgaaagcgctcgccagcagcggcccggcggaggacggctggctcgataacagcctcgacgagcgccagcggctgatcgccgccctggaaaaagcgggctgggtgcaggccaaagcggcgcggctgctcggcatgaccccgcgccaggtggcgtatcgcattcagattatggatatcaccatgccgcgactgtgaagccttatgtgagattcaggacattgtcgccagcgcggcggaattgcgacaattcagggacgcgggttgccggttaaaaagtctacttttcatgcggttgcgaaattaacctctggtacagcatttgcagcaggaaggtatcgcccaaccacgaaggtacgaccatgacttcctgctcctctttttctggcggcaaagcctgccgcccggcggatgacagcgcattgacgccgcttgtggccgataaagctgccgcgcacccctgctactctcgccatgggcatcaccgtttcgcgcggatgcatctgcccgtcgcgcccgcctgcaatttgcagtgcaactactgtaatcgcaaattcgattgcagcaacgagtcccgccccggggtatcgtcaacgctgctgacgcctgaacaggcggtcgtgaaagtgcgtcaggtcgcgcaggcgatcccgcagctttcggtggtgggcatcgccgggcccggcgatccgctcgccaatatcgcccgcacctttcgcaccctggagctgatccgcgaacagctgccggacctgaaattatgcctgtcgaccaacggactggtgctgcctgacgcggtggaccgcctgctggatgtcggcgttgaccacgtcacggtcaccattaacaccctcgacgcggagattgccgcgcaaatctacgcctggctatggctggacggcgaacgctacagcgggcgcgaagcgggagagatcctgattgcccgtcagcttgagggcgtacgcaggctgaccgccaaaggcgtgctggtgaaaataaattcggtgctgatccccggtatcaacgatagcggcatggccggcgtgagccgcgcgctgcgggccagcggcgcgtttatccataatattatgccgctgatcgccaggccggagcacggcacggtgtttggcctcaacggccagccggagccggacgccgagacgctcgccgccacccgcagccggtgcggcgaagtgatgccgcagatgacccactgccaccagtgtcgcgccgacgccattgggatgctcggcgaagaccgcagccagcagtttacccagcttccggcgccagagagtctcccggcctggctgccgatcctccaccagcgcgcgcagctgcacgccagcattgcgacccgcggcgaatctgaagccgatgacgcctgcctggtcgccgtggcgtcaagccgcggggacgtcattgattgtcactttggtcacgccgaccggttctacatttacagcctctcggccgccggtatggtgctggtcaacgagcgctttacgcccaaatattgtcaggggcgcgatgactgcgagccgcaggataacgcagcccggtttgcggcgatcctcgaactgctggcggacgttaaagccgtattctgcgtgcgtatcggccatacgccgtggcaacagctggaacaggaaggcattgaaccctgcgttgacggcgcgtggcggccggtctccgaagtgctgcccgcgtggtggcaacagcgtcgggggagctggcctgccgcgttgccgcataagggggtcgcctgatgccgccgctcgactggttgcggcgcttatggctgctgtaccacgcggggaaaggcagctttccgctgcgcatggggcttagcccgcgcgattggcaggcgctgcggcggcgcctgggcgaggtggaaacgccgctcgacggcgagacgctcacccgtcgccgcctgatggcggagctcaacgccacccgcgaagaggagcgccagcagctgggcgcctggctggcgggctggatgcagcaggatgccgggccgatggcgcagattatcgccgaggtttcgctggcgtttaaccatctctggcaggatcttggtctggcatcgcgcgccgaattgcgcctgctgatgagcgactgctttccacagctggtggtgatgaacgaacacaatatgcgctggaaaaagttcttttatcgtcagcgctgtttgctgcaacagggggaagttatctgccgttcgccaagctgcgacgagtgctgggaacgcagcgcctgttttgagtagccgtttcccgaagggggcgctgcaaacaaaaagccggaggtttccctccggcttttcacatcatcaaatgtgattatgcgacgtcttcgtactgcggcaccgggttgcggaagcttttggtcacgcaggcctccgtagaccagaccaataccgccccagatcaggccgagaaccatggagctctcttcgaggttaatccacagtgcgccgacggtcagcgcgccgcagaccggcagaatcagatagttgaagtggtctttcagcgttttgttgcgcttttcacggatccagaactgggagatcaccgacaggttaacgaaggtgaacgccaccagcgcgccgaggttaatcggcgccgtcgccgtgacgaggtcgagtttaatcgccagcagcgcgatcgcgcaaccagcagcacgttccatgccggagtacgccgtttcggatgcacgtagccgaagaaacgcgtcgggaacacgccgtcgcggcccatcacgtacatcagacgggaaacgcccgcgtgcgcggccgtgccggatgccagtacggtaacgctggagaaaatcagcacgccccactggaaggttttgcccgccacgtacagcatgatttcaggctgcgaggcgtccggatctttgaagcgcgagatgtccgggaagtacagctgcag